(19) United States
(12) Patent Application Publication
(10) Pub. No.: US 2025/0283896 A1
(43) Pub. Date: Sep. 11, 2025

(54) PROBES AND METHODS FOR TARGETED VISUALIZATION OF NLRP3 INFLAMMASOMES

(71) Applicant: VANDERBILT UNIVERSITY, Nashville, TN (US)

(72) Inventors: Imam UDDIN, Nashville, TN (US); Marcell PAGUAGA, Nashville, TN (US)

(21) Appl. No.: 18/859,862

(22) PCT Filed: Apr. 25, 2023
(86) PCT No.: PCT/US2023/019803

§ 371 Date: Oct. 24, 2024

Related U.S. Application Data
(60) Provisional application No. 63/334,384, filed on Apr. 25, 2022.

(51) Int. Cl. G01N 33/68; G01N 33/58

(52) U.S. Cl. CPC G01N 33/6893; G01N 33/582; G01N 2800/164; G01N 2800/7095

(57) ABSTRACT

The present disclosure relates to probes and methods for detecting and imaging ocular diseases.

### RELATED APPLICATION

This PCT application claims priority to, and the benefit of, U.S. Provisional Patent Application No. 63/334,384, filed Apr. 25, 2022, entitled “PROBES AND METHODS FOR TARGETED VISUALIZATION OF NLRP3 INFLAMMASOMES,” which is incorporated by reference herein in its entirety.

### STATEMENT REGARDING FEDERALLY SPONSORED RESEARCH

This invention was made with Government Support under Grant Nos. EY029693 and EY023397, awarded by the National Institutes of Health. The Government has certain rights in the invention.

### FIELD

The present disclosure relates to probes and methods for detecting and imaging ocular diseases.

### BACKGROUND

Wet form of age-related macular degeneration (wet AMD) is a progressive vascular disease that mainly affects older adults and causes severe and irreversible vision loss. A key complication that contributes to wet AMD progression is choroidal neovascularization (CNV). The initiation and progression of CNV may be regulated by vascular endothelial growth factor (VEGF), and indeed anti-VEGF therapy is highly effective for the management of this vascular disease. However, many wet AMD patients do not respond to this therapy, causing a major challenge to their clinicians. Resistance to anti-VEGF treatments may reflect the existence of other mediators of this disease. For example, activated monocytes migrate to the site of choroidal neovascular lesions where they become macrophages and induce inflammation, possibly through a mechanism mediated by the NLRP3 inflammasome. Since activated NLRP3 is correlated with inflammation in CNV, visualizing NLRP3 inflammasomes and their associated macrophages is of great interest to monitor wet AMD progression and develop effective therapies against it. However, current ophthalmic imaging systems do not permit such targeted imaging.

Due to the association between NLRP3-mediated inflammation, activated macrophages, and CNV, an NLRP3-targeted fluorescent probe was used to visualize pro-inflammatory macrophages in CNV. Although an NLRP3-targeted fluorescent probe was synthesized by conjugating a fluorophore, coumarin 343, to the NLRP3 inhibitor, MCC950, this probe was not suitable for ophthalmic in vivo applications. Thus there is a need for an in vivo ophthalmic probe. The compounds, compositions, and methods disclosed herein address these and other needs.

### SUMMARY

In accordance with the purposes of the disclosed materials and methods, as embodied and broadly described herein, the disclosed subject matter, in one aspect, relates to probes, imaging, and detection methods for monitoring ocular diseases.

Thus, in one aspect disclosed herein is a probe comprising a compound having a formula

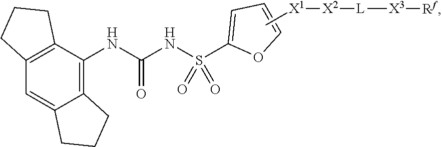

or a salt thereof, wherein X¹ is selected from null, O, NH, C(═O), C(═O)NH, C(═O)O, NHC(═O)NH, NHC(═O)O, C₁₋₂alkyl, X² is selected from null, O, NH, C(═O), C(═O)NH, C(═O)O, NHC(═O)NH, NHC(═O)O, C₁₋₂alkyl, X³ is selected from null, O, NH, C(═O), C(═O)NH, C(═O)O, NHC(═O)NH, NHC(═O)O, C₁₋₂alkyl, L is null or a linker, and R^{f} is a fluorophore.

In some embodiments, the probe comprises a compound having the formula

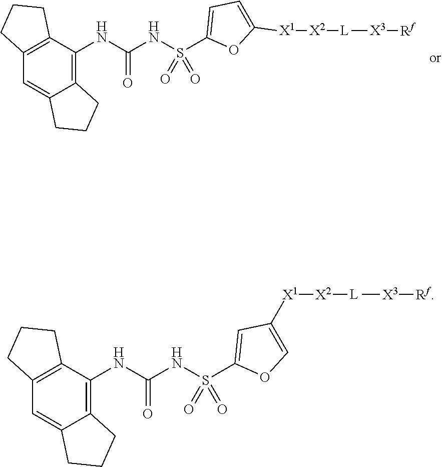

In some embodiments, X¹ is null, CH₂, or C(CH₃)₂. In some embodiments, X¹ is null. In some embodiments, X¹ is CH₂. In some embodiments, X¹ is C(CH₃)₂. In some embodiments, X² is C(═O)NH or C(═O)O. In some embodiments, X² is C(═O)NH. In some embodiments, X² is C(═O)O. In some embodiments, L is null or C₁₋₈alkylene. In some embodiments, L is null. In some embodiments, L is C₁₋₈alkylene. In some embodiments, L is C₂₋₆alkylene or C₄₋₆alkylene. In some embodiments, X³ is null, NH, O, C(═O)NH, C(═O)O, NHC(═O)NH, or NHC(═O)O. In some embodiments, X³ is null. In some embodiments, X³ is NH. In some embodiments, X³ is O. In some embodiments, X³ is C(═O)O. In some embodiments, X³ is C(═O)NH.

In some embodiments, R^{f} is a xanthene fluorophore, BODIPY fluorophore, a cyanine fluorophore, a coumarin fluorophore, or an azo fluorophore. In some embodiments, R^{f} is a xanthene fluorophore selected from a rhodamine fluorophore, or a fluorescein fluorophore.

In some embodiments, R^{f} has the formula

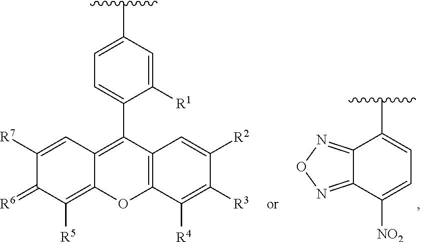

wherein R¹ is H, CO₂H, or CO₂C₁₋₄alkyl, R² is H or halo, R⁴ is H or halo, R⁵ is H or halo, R⁷ is H or halo, R³ is OH or N(Rⁿ)₂, wherein Rⁿ is in each case independently selected from H or C₁₋₄alkyl, and R⁶ is O or N⁺(Rⁿ)₂, wherein Rⁿ is in each case independently selected from H or C₁₋₄alkyl.

In some embodiments, R^{f} has the formula:

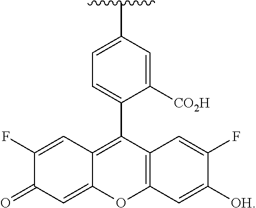 [SMILES: *c1ccc(-c2c3cc(F)c(=O)cc-3oc3cc(O)c(F)cc23)c(C(=O)O)c1]

In some embodiments, R^{f} is fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.), ALEXA FLUOR™ 350, ALEXA FLUOR™ 405, ALEXA FLUOR™ 430, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, Pacific Orange, rhodamine 6G, rhodamine green, rhodamine red, tetramethyl rhodamine, Texas Red (available from Molecular Probes, Inc., Eugene, Oreg.), PE-Texas Red, APC-Cy7, PE-Alexa dyes (610, 647, and 680), or APC-Alexa dyes.

In one aspect, disclosed herein is a method of detecting an ocular disease in a subject comprising administering to the subject the probe of any preceding aspect. In some embodiments, the ocular disease is age-related macular degeneration (AMD), retinopathy of prematurity (ROP), diabetic retinopathy (DR), or branch retinal vein occlusion (BRVO).

In some embodiments, the probe detects an activated NLRP3-mediated inflammasome. In some embodiments, the probe detects a pro-inflammatory macrophage.

In one aspect, disclosed herein is a method for ocular imaging comprising administering to a subject the probe of any preceding aspect. In some embodiments, the probe is administered to the subject in combination with a retinal imaging system. In some embodiments, the probe emits a fluorescent signal following exposure to a light stimulus. In some embodiments, the light stimulus is a laser source.

In some embodiments, the probe comprises an NLRP3 inhibitor. In some embodiments, the NLRP3 inhibitor comprises an MCC-950 inhibitor.

In some embodiments, the subject is a mammal.

### BRIEF DESCRIPTION OF FIGURES

The accompanying figures, which are incorporated in and constitute a part of this specification, illustrate several aspects described below.

FIG. 1 is a graphical abstract of InflammaProbe-1, which is composed of a selective NLRP3 inhibitor conjugated to a fluorophore, as a method to detect and image ocular disease progression.

FIG. 2 shows the design and synthesis of InflammaProbe-1. InflammaProbe-1 was synthesized by conjugating a selective inhibitor of NLRP3 (CY09) to a commercially available fluorophore (Oregon Green® 488). Conjugation was achieved with an EDCI-mediated coupling method.

FIGS. 3A-3B show that InflammaProbe-1 can inhibit NLRP3-mediated secretion of IL-1β but not TNF-α. (FIG. 3A) InflammaProbe-1 and CY-09 dose-dependently inhibited NLRP3-mediated secretion of IL-1β in LPS-primed and nigericin-stimulated murine bone marrow-derived macrophages. (FIG. 3B) InflammaProbe-1 and CY-09 had no significant effect on LPS-induced secretion of TNF-α in comparison to the LPS and nigericin control, except for CY-09 at 10 μM. These data suggest that InflammaProbe-1 retains the inhibitory ability of its parent compound, CY-09, that enables it to target the NLRP3 inflammasome. Levels of IL-1β and TNF-α were measured by performing ELISA. The data were expressed as the mean±SD (n=3).

FIGS. 4A-4I show in vitro imaging of NLRP3 in BMDMs using InflammaProbe-1. FIGS. 4A-4C show untreated BMDMs, FIGS. 4D-4F show LPS-primed BMDMs, and FIGS. 4G-4I show LPS-primed and nigericin-stimulated BMDMs were stained with 10 μM InflammaProbe-1, fixed on microscope slides, and imaged using confocal fluorescence microscopy. Inflammaprobe-1-dependent fluorescence was clearly observed in LPS-primed cells (FIGS. 4D-4F) and in LPS-primed and nigericin stimulated cells (FIGS. 4G-4I), but not in untreated cells (FIGS. 4A-4C). These results indicate that InflammaProbe-1 selectively stains macrophages that have been induced to express NLRP3. These are representative images of four replicates from each experimental group.

FIGS. 5A-5D show in vivo imaging of NLRP3 in LCNV using InflammaProbe-1. FIGS. 5A-5C show brightfield and FIGS. 5B-5D show fluorescence fundus images of murine laser-induced choroidal neovascularization (LCNV) taken 6 h after a 10 mg/kg intraperitoneal injection of InflammaProbe-1 on day 4 post LCNV. The fluorescence fundus image clearly shows InflammaProbe-1-dependent fluorescence that is localized exclusively to each of the LCNV lesions observed in the brightfield fundus image. These are representative images of 12 murine LCNV eyes.

FIGS. 6A-6H show ex vivo imaging of NLRP3 in LCNV using InflammaProbe-1. Four days after laser-induced choroidal neovascularization (LCNV), mice were intraperitoneally injected with InflammaProbe-1 at 10 mg/kg and enucleated after 6 h. Their choroids were dissected and co-stained with fluorescently tagged antibodies against IBA1, which targets macrophages, and IB4, which stains primarily endothelial cells. The stained choroidal lesions were then imaged with confocal fluorescence microscopy at (FIGS. 6A-6D) 10× magnification and (FIGS. 6E-6H) 63× magnification. The white arrows indicate endothelial cells (cells that are IB4⁺ but IBA1⁻ and InflammaProbe-1⁻). Areas of overlap between at least two stains appear white. These are representative images taken from 12 choroids.

FIGS. 7A-7D show three-dimensional reconstruction of ex vivo LCNV lesions and correlation of InflammaProbe-1⁺, IBA1⁺, and IB4⁺ cells. Three-dimensional reconstruction of the stained LCNV lesion (FIGS. 7A-7D) using Z-stacked confocal fluorescent images at 63× magnification. Areas of overlap between at least two stains appear white. The degree of correlation within each pair of stains is indicated by Pearson's correlation coefficient (r).

FIGS. 8A-8B show toxicity of InflammaProbe-1. (FIG. 8A) Cytotoxicity of InflammaProbe-1 was assessed in primary mouse retinal microvascular endothelial cells (MRMEC) using a fluorescence-based assay with Calcein Deep Red™ AM ester. The viability of MRMECs was not significantly reduced by a 20-h exposure to 1-20 μM InflammaProbe-1 in comparison to untreated cells. (FIG. 8B) Retinal toxicity was assessed in dark-adapted mice using ganzfeld electroretinography (ERG) 7 days after an intraperitoneal injection of InflammaProbe-1 at 10 mg/kg. Relative to the retinas of mice injected with saline or a vehicle control (10% DMSO in PBS), the retinas of mice that had been injected with InflammaProbe-1 did not show any significant reduction in the a-wave and b-wave amplitudes of their electrical response to a 1 Log cd s/m² light flash. These results suggest that InflammaProbe-1 is not toxic to primary cells or retinal tissues. Cell viability data were expressed as the mean±SD of 6 replicates from each group. Statistical analysis by unpaired t-tests with Welch's corrections.

FIG. 9 is a high-resolution mass spectrum (HRMS) of InflammaProbe-1.

FIG. 10 is an excitation and emission spectra of InflammaProbe-1.

FIG. 11 shows nuclear magnetic resonance (NMR) spectra of InflammaProbe-1.

FIG. 12 shows an expanded view of the NMR spectra of InflammaProbe-1 in FIG. 11 between 6.0 and 9.0 ppm.

FIG. 13 shows in vivo retinal toxicity of InflammaProbe-1. Adult C57BL/6 mice were injected intraperitoneally with InflammaProbe-1 at 10 mg/kg body weight, vehicle control (10% DMSO in PBS), or saline control. Seven days later, retinal toxicity was assessed in the dark-adapted mice using ganzfeld electroretinography (ERG). The retinas were stimulated with light flashes ranging from −4 to 2 Log cd·s/m² and their electrical responses were recorded. Data (A-B) were expressed as mean±SD of 4 retinas per group.

FIGS. 14A, 14B, 14C, 14D, and 14E show an SS-32 compound. FIG. 14A shows the SS-32 compound with a 4^{th} position methanamine-OG. FIGS. 14B and 14C show Liquid Chromatography/Mass Spectrometry (LC/MS) spectra with low resolution (ESI+): m/z [M+H]⁺ calculated for C₃₉H₂₉F₂N₃O₁₀S, 769.1542; Found, 769.5, 770.5, and 771.5. FIGS. 14D and 14E show High-Resolution Mass Spectrometry (HRMS) spectra with (ESI−): m/z [M−H]⁺ calculated for C₃₉H₂₉F₂N₃O₁₀S, 769.1542; Found, 768.1511.

FIGS. 15A, 15B, 15C, and 15D show an MI-146 compound. FIG. 15A shows the MI-146 compound with a 3^{rd} position-OG conjugate. FIGS. 15B, 15C, and 15D show LC/MS spectra with low resolution (ESI+): m/z [M+H]⁺ calculated for C₄₄H₃₈F₂N₄O₁₁S, 868.2226; Found, 866.5, 867.5, and 868.5.

FIGS. 16A, 16B, 16C, 16D, and 16E show an SS-15 synthetic intermediate compound. FIG. 16A shows the SS-15 synthetic intermediate with a 4^{th} position Boc protected drug. FIGS. 16B and 16C show LC/MS spectra with low resolution (ESI+): m/z [M+H]⁺ calculated for C₂₃H₂₉N₃O₆S, 475.1777; Found, 475.2, 475.5, and 476.1. FIG. 16D shows HRMS spectra with (ESI−): m/z [M−H]⁺ calculated for C₂₃H₂₉N₃O₆S, 475.1777; Found, 474.1685. FIG. 16E shows a proton Nuclear Magnetic Resonance (¹H NMR) spectra with ¹H NMR (600 MHZ, DMSO D6) δ 7.37 (s, 1H), 6.81 (d, J=2.76 Hz, 1H), 6.2 (d, J=3.22 Hz, 1H), 4.10 (s, 2H), 2.74 (t, J=7.28 Hz, 4H), 2.62 (t, J=7.26 Hz, 4H), 1.93-1.88 (m, J=7.35 Hz, 4H).

FIGS. 17A, 17B, 17C, 17D, and 17E show an SS-31 synthetic intermediate compound. FIG. 17A shows the SS-31 synthetic intermediate compound with a 4^{th} position methanamine drug (or 4th position free amine). FIGS. 17B and 17C show LC/MS spectra with low resolution (ESI+): m/z [M+H]⁺ calculated for C₁₈H₂₁N₃O₄S, 375.1252; Found, 376.1 and 751.3, 752.4, 753.3 as compound dimer. FIG. 17D shows HRMS spectra with (ESI−): m/z [M−H]⁺ calculated for C₁₈H₂₁N₃O₄S, 375.1252; Found, 374.1180. FIG. 17E shows ¹H NMR spectra with ¹H NMR (600 MHZ, DMSO D6) δ 6.79 (s, 1H), 6.62 (d, J=2.76 Hz, 1H), 6.5 (d, J=3.22 Hz, 1H), 4.07 (s, 1H), 2.76 (t, J=7.28 Hz, 4H), 2.67 (t, J=7.26 Hz, 4H), 1.94-1.89 (m, J=7.32 Hz, 4H).

FIGS. 18A, 18B, 18C, 18D, and 18E show an SS-01 synthetic intermediate compound. FIG. 18A shows the SS-01 synthetic intermediate compound with a 3^{rd} position carboxylate. FIGS. 18B and 18C show LC/MS spectra with low resolution (ESI+): m/z [M+H]⁺ calculated for C₂₀H₂₂N₂O₆S, 418.1199; Found, 419.1, 420.2 and 859.4 as compound dimer. FIG. 18D shows HRMS spectra with (ESI−): m/z [M−H]⁺ calculated for C₂₀H₂₂N₂O₆S, 418.1199; Found, 417.1139. FIG. 18E shows 1H NMR spectra with ¹H NMR (600 MHz, DMSO D6) δ 8.4 (s, 1H), 6.88 (s, 1H), 6.81 (s, 1H), 4.26 (m, J=6.89 Hz, 2H), 2.77 (t, J=7.36 Hz, 4H), 2.65 (t, J=7.36 Hz, 4H), 1.9 (m, J=7.38 Hz, 4H), 1.27 (t, J=7.21 Hz, 3H).

FIGS. 19A, 19B, 19C, 19D, 19E, and 19F show an SS-03 synthetic intermediate compound. FIG. 19A shows the SS-03 synthetic intermediate compound with a 3^{rd} position carboxylic acid. FIGS. 19B, 19C, and 19D show LC/MS spectra with low resolution (ESI+): m/z [M+H]⁺ calculated for C₁₈H₁₈N₂O₆S, 390.0886; Found, 391.1, 392.1 and 803.3 as compound dimer. FIG. 19E shows HRMS spectra with (ESI−): m/z [M−H]⁺ calculated for C₁₈H₁₈N₂O₆S, 390.0886; Found, 389.0824. FIG. 19F shows ¹H NMR spectra with ¹H NMR (600 MHZ, DMSO D6) δ 7.91 (s, 1H), 6.78 (s, 1H), 6.67 (s, 1H), 2.74 (t, J=7.42 Hz, 4H), 2.65 (t, J=7.42 Hz, 4H), 2.49 (m, J=7.38 Hz, 4H).

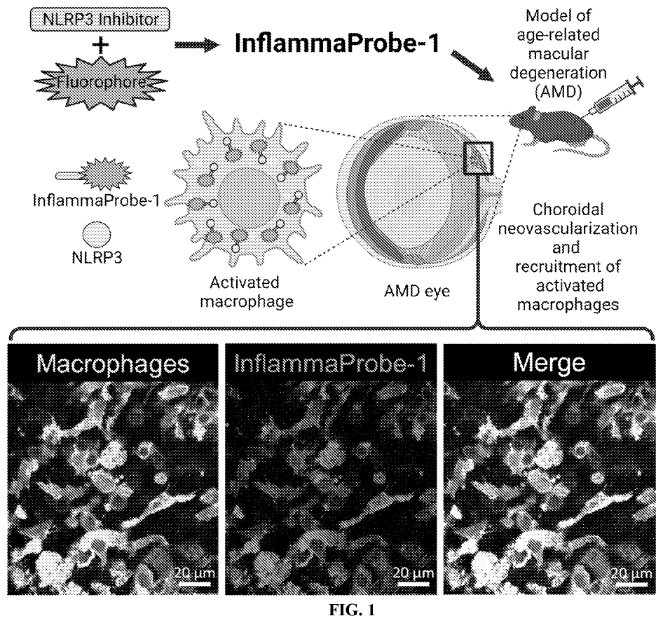

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

The term “subject” can refer to a human in need of treatment for any purpose, and more specifically a human in need of such a treatment to treat age-related macular degeneration. However, the term “subject” can also refer to non-human animals, for example mammals such as dogs, cats, horses, cows, pigs, sheep, and non-human primates, among others, that are in need of treatment.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, the term “chemical compound” or “compound” refers to a chemical substance consisting of two or more different types of atoms or chemical elements in a fixed stoichiometric proportion. These compounds have a unique and defined chemical structure held together in a defined spatial arrangement by chemical bonds. Chemical compounds can be held together by covalent bonds, ionic bonds, metallic ions, or coordinate covalent bonds.

The term “detect” or “detecting” refers to an output signal released for the purpose of sensing of physical phenomenon. An event or change in environment is sensed and signal output released in the form of light.

As used herein, “cytotoxicity” refers to the quality of being toxic to cells. Treating cells with a cytotoxic compound can result in a variety of cell fates, including necrosis (in which the cell membrane becomes compromised leading to cell lysis), senescence (in which the cell stops actively growing and dividing), or apoptosis (in which the cell activates a genetic program of controlled cell death).

As used herein, the term “ophthalmic” refers to any study, procedure, treatment, and anatomical structure pertaining to the eye. The term “ocular” refers to the eyes, which are the major organs of the visual system.

The “retina” is the innermost, light-sensitive layer of tissue within the eye of most vertebrates, including, but not limited to humans. Retinal tissue comprises several layers made up of light-sensing cells called photoreceptor cells, which detect and process light coming into the retina.

The “macula” refers to an oval-shaped pigmented area in the center of the retina of most vertebrate eyes, including, but not limited to humans. This area of the retina is responsible for producing central, high-resolution color vision. High-resolution color vision is lost when the macula is damaged as a result of macular degeneration.

The “fovea” refers to the more centrally located region within the macula of the retina of most vertebrates, including, but not limited to humans. The fovea is a small, central locus of densely packed photoreceptor cells, called cones, responsible for sharp, central vision.

The term “photoreceptor” refers to a cell or protein within the eye that responds to light stimuli. A photoreceptor cell is a specialized cell type found in the retina that is capable of visual phototransduction, or the ability to convert light into signals that can stimulate biological processes. A photoreceptor protein is a light-sensitive protein found in photoreceptor cells involved in sensing and responding to light stimuli. Specifically, these proteins absorb light molecules called photons, triggering a change in the cell's membrane potential.

As used herein, unless otherwise indicated, the terms “alkylene”, “alkenylene”, “cycloalkylene” and “cycloalkenylene” refer to a divalent hydrocarbon radical that is formed by removal of a hydrogen atom from an alkyl, alkenyl, cycloalkyl or cycloalkenyl radical, respectively, as such terms are defined above.

Affixing the suffix “-ene” to a group indicates the group is a polyvalent moiety, e.g., boned to two or more groups. Alkylene is the polyvalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

As used herein, the designation of a polyvalent moiety without specifying the specific order of attachment is intended to cover all possible arrangements. By way of example, a compound represented by the formula A-X—B, wherein X is NHC(═O) embraces both:

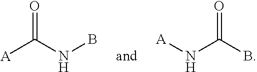

As used herein, a chemical bond depicted [symbol] represents either a single, double, or triple bond, valency permitting. By way of example,

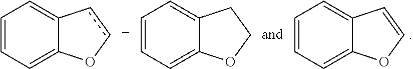

Unless stated to the contrary, a substituent drawn without explicitly specifying the point of attachment indicates that the substituent may be attached at any possible atom. For example, in a benzofuran depicted as

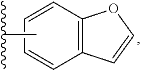

the substituent may be present at any one of the six possible carbon atoms.

### Compounds and Compositions

In one aspect disclosed herein is a probe comprising a compound having a formula

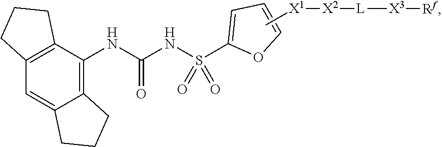

or a salt thereof, wherein X¹ is selected from null, O, NH, C(═O), C(═O)NH, C(═O)O, NHC(═O)NH, NHC(═O)O or C₁₋₂alkyl, X² is selected from null, O, NH, C(═O), C(═O)NH, C(═O)O, NHC(═O)NH, NHC(═O)O or C₁₋₂alkyl, X³ is selected from null, O, NH, C(═O), C(═O)NH, C(═O)O, NHC(═O)NH, NHC(═O)O or C₁₋₂alkyl, L is null or a linker, and R^{f} is a fluorophore.

As used herein, the term “probe” refers to a molecule or group of molecules used in molecular biology or chemistry to study the properties of other molecules or structures. If some measurable property of the molecular probe used changes when it interacts with the molecule of interest, the interactions between the probe and the molecule of interest can be studied. This makes it possible to indirectly study the properties of compounds and structures which may be hard to study directly.

As used herein, “fluorophore” is a fluorescent chemical compound that can re-emit light upon light excitation. The chemicals are sometimes used alone as a tracer in fluids, as a due for staining certain structures, as an enzyme substrate, or as a probe/indicator. More commonly they are covalently bonded to a macromolecule to serve as a marker for bioactive reagents (e.g., antibodies, peptides, nucleic acids, etc.) Fluorophores are notably used to stain tissues, cells, or materials in a variety of analytical methods such as fluorescent imaging and spectroscopy. Fluorophores can be divided into two main classes—intrinsic and extrinsic. Intrinsic fluorophores occur naturally and include, but are not limited to, aromatic amino acids, NADH, flavins, derivatives of pyridoxyl, and chlorophyll. Extrinsic fluorophores can be added to a sample to provide fluorescence when none exists, or to change the spectral properties of a sample, and include, but are not limited to, dansyl, fluorescein, and rhodamine. Further, fluorophores can include 4-nitrobenzofurazan.

In some embodiments, the probe comprises a compound having the formula

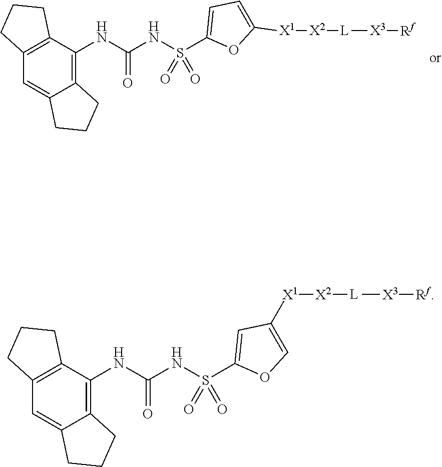

In some embodiments, X¹ is null, CH₂, or C(CH₃)₂. In some embodiments, X¹ is null. In some embodiments, X¹ is CH₂. In some embodiments, X¹ is C(CH₃)₂. In some embodiments, X² is C(═O)NH or C(═O)O. In some embodiments, X² is C(═O)NH. In some embodiments, X² is C(═O)O. In some embodiments, L is null or C₁₋₈alkylene. In some embodiments, L is null. In some embodiments, L is C₁₋₈alkylene. In some embodiments, L is C₂₋₆alkylene or C₄₋₆alkylene. In some embodiments, X³ is null, NH, O, C(═O)NH, C(═O)O, NHC(═O)NH, or NHC(═O)O. In some embodiments, X³ is null. In some embodiments, X³ is NH. In some embodiments, X³ is O. In some embodiments, X³ is C(═O)O. In some embodiments, X³ is C(═O)NH.

In some embodiments, R^{f} is a xanthene fluorophore, BODIPY fluorophore, a cyanine fluorophore, a coumarin fluorophore, an azo fluorophore, or a 4-nitrobenzofurazan fluorophore. In some embodiments, R^{f} is a xanthene fluorophore selected from a rhodamine fluorophore and a fluorescein fluorophore.

In some embodiments, R^{f} has the formula

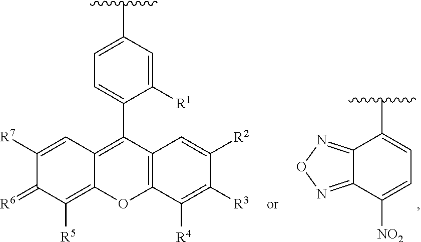

wherein R¹ is H, CO₂H, or CO₂C₁₋₄alkyl, R² is H or halo, R⁴ is H or halo, R⁵ is H or halo, R⁷ is H or halo, R³ is OH or N(Rⁿ)₂, wherein Rⁿ is in each case independently selected from H, C₁₋₄alkyl, R⁶ is O or N⁺(Rⁿ)₂, wherein Rⁿ is in each case independently selected from H or C₁₋₄alkyl.

In some embodiments, R^{f} has the formula:

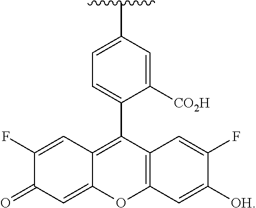 [SMILES: *c1ccc(-c2c3cc(F)c(=O)cc-3oc3cc(O)c(F)cc23)c(C(=O)O)c1]

In some embodiments, R^{f} is fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.), ALEXA FLUOR™ 350, ALEXA FLUOR™ 405, ALEXA FLUOR™ 430, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, Pacific Orange, rhodamine 6G, rhodamine green, rhodamine red, tetramethyl rhodamine, Texas Red (available from Molecular Probes, Inc., Eugene, Oreg.), PE-Texas Red, APC-Cy7, PE-Alexa dyes (610, 647, and 680), or APC-Alexa dyes.

In some embodiments, the probe comprises a compound having the formula

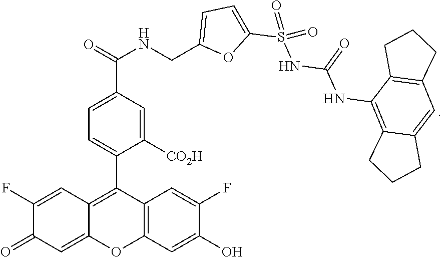 [SMILES: O=C(Nc1c2c(cc3c1CCC3)CCC2)NS(=O)(=O)c1ccc(CNC(=O)c2ccc(-c3c4cc(F)c(=O)cc-4oc4cc(O)c(F)cc34)c(C(=O)O)c2)o1]

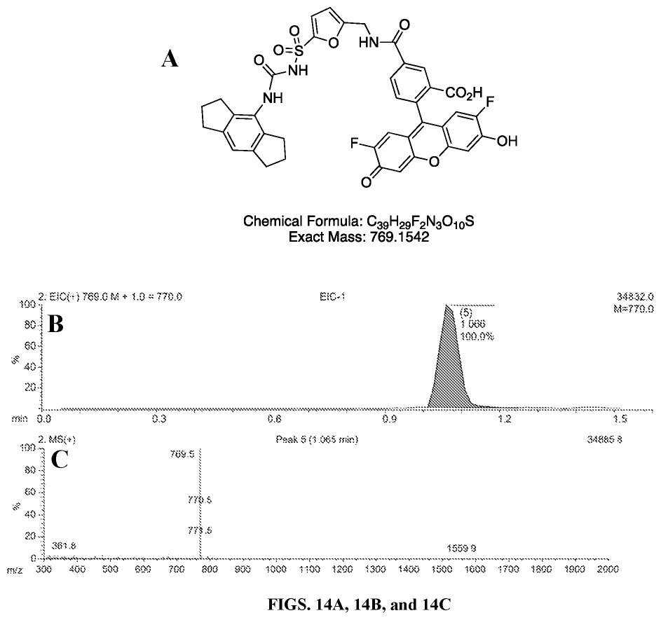

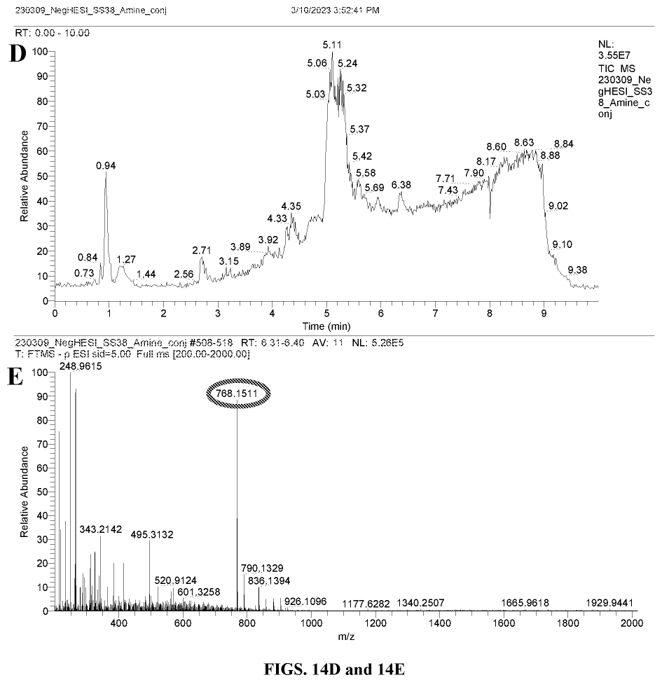

In some embodiments, the probe comprises a SS-32 compound (FIGS. 14A-14E).

In some embodiments, the probe comprises a compound having the formula

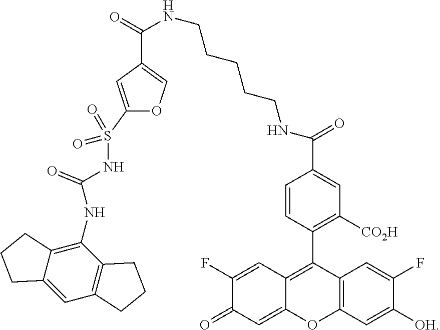 [SMILES: O=C(Nc1c2c(cc3c1CCC3)CCC2)NS(=O)(=O)c1cc(C(=O)NCCCCCNC(=O)c2ccc(-c3c4cc(F)c(=O)cc-4oc4cc(O)c(F)cc34)c(C(=O)O)c2)co1]

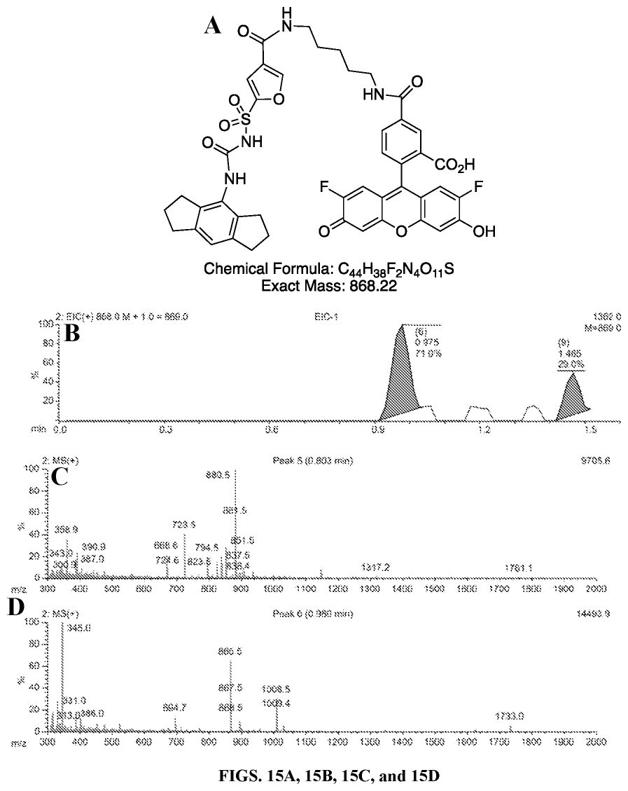

In some embodiments, the probe comprises an MI-146 compound (FIGS. 15A-15D).

In some embodiments, the probe can comprise a compound having the formula

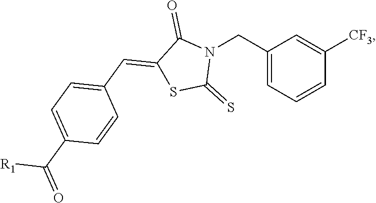

wherein R₁ is a fluorophore or R^{f} of any preceding aspect.

In some embodiments, the probe can include, but is not limited to any one of the following formulas:

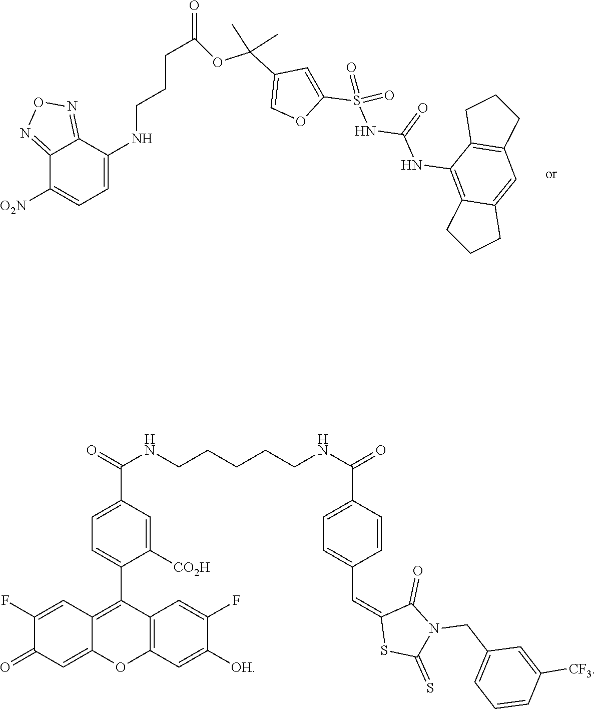 [SMILES: CC(C)(OC(=O)CCCNc1ccc([N+](=O)[O-])c2nonc12)c1coc(S(=O)(=O)NC(=O)Nc2c3c(cc4c2CCC4)CCC3)c1 ; O=C(NCCCCCNC(=O)c1ccc(-c2c3cc(F)c(=O)cc-3oc3cc(O)c(F)cc23)c(C(=O)O)c1)c1ccc(/C=C2/SC(=S)N(Cc3cccc(C(F)(F)F)c3)C2=O)cc1]

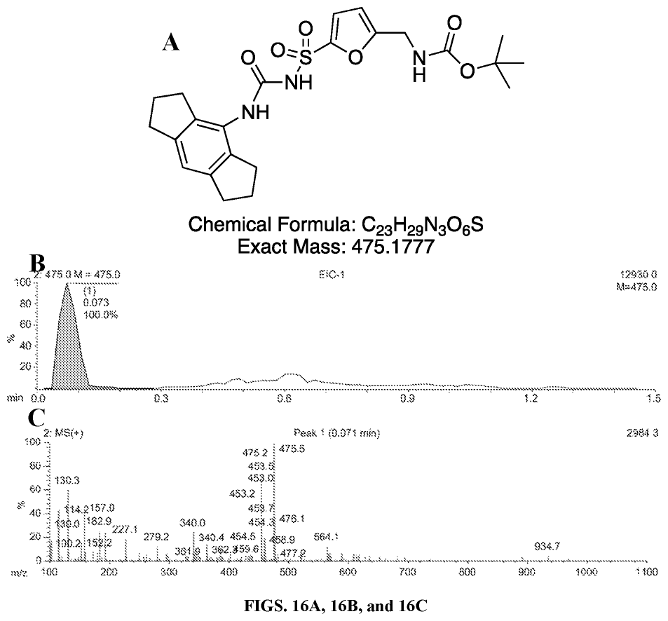

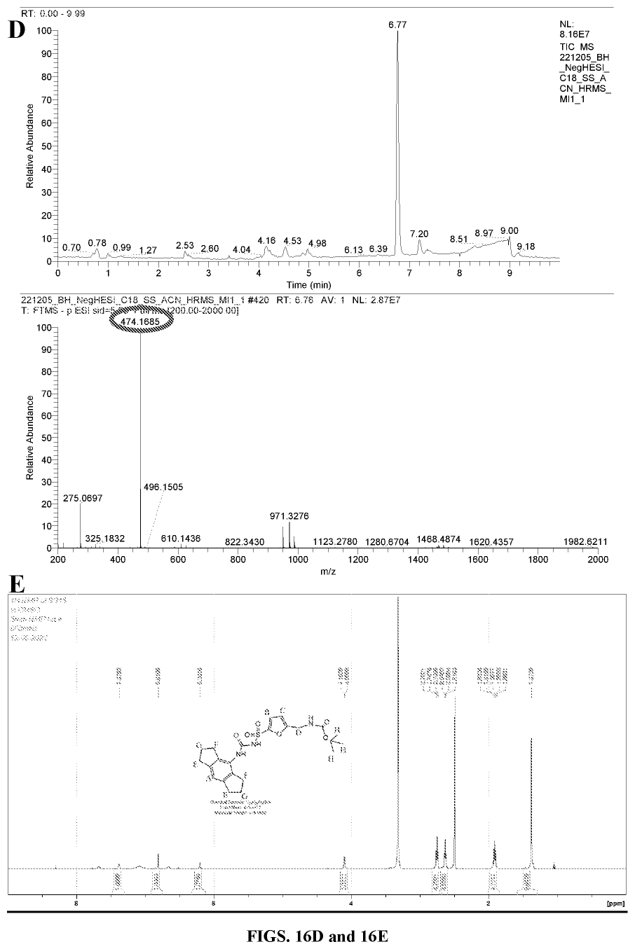

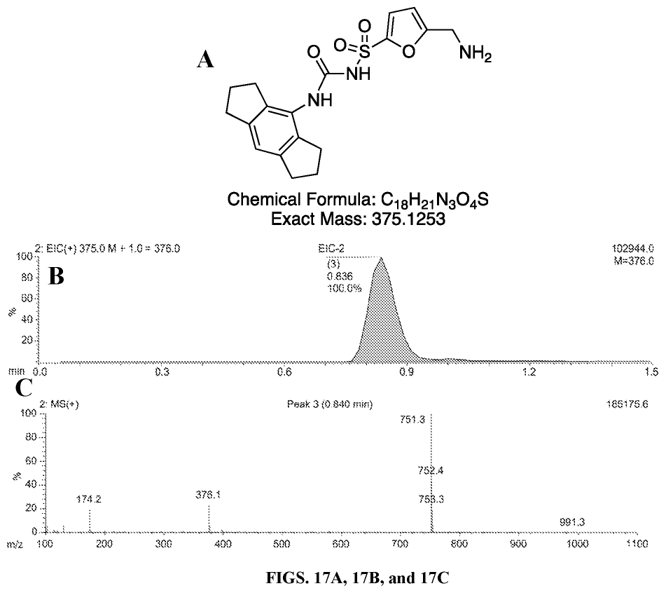

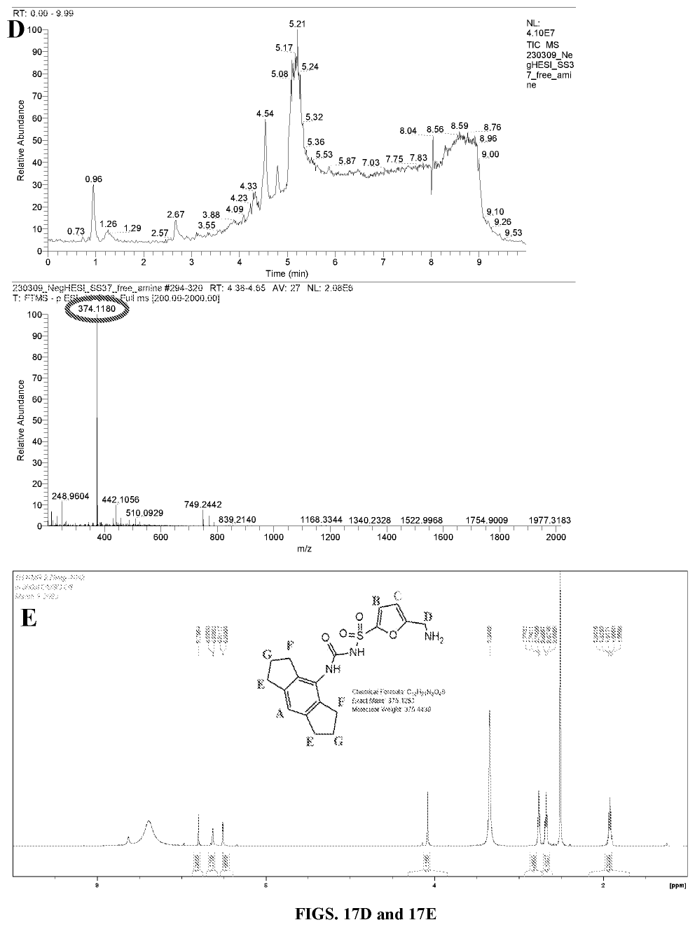

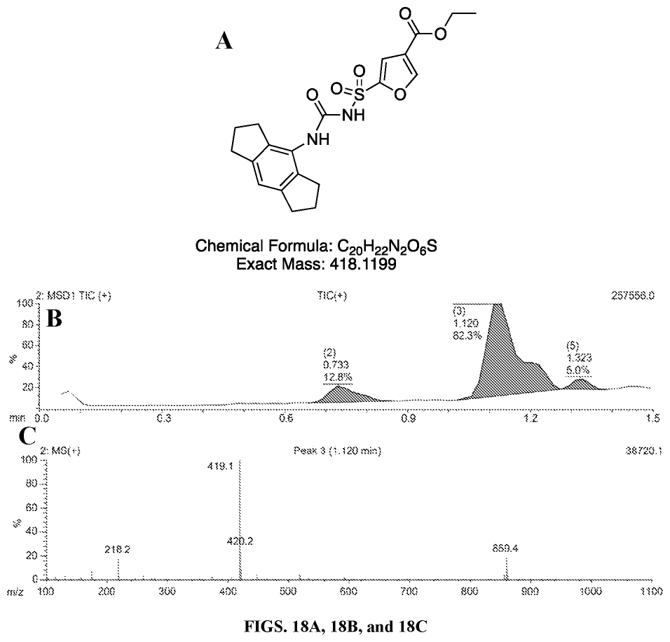

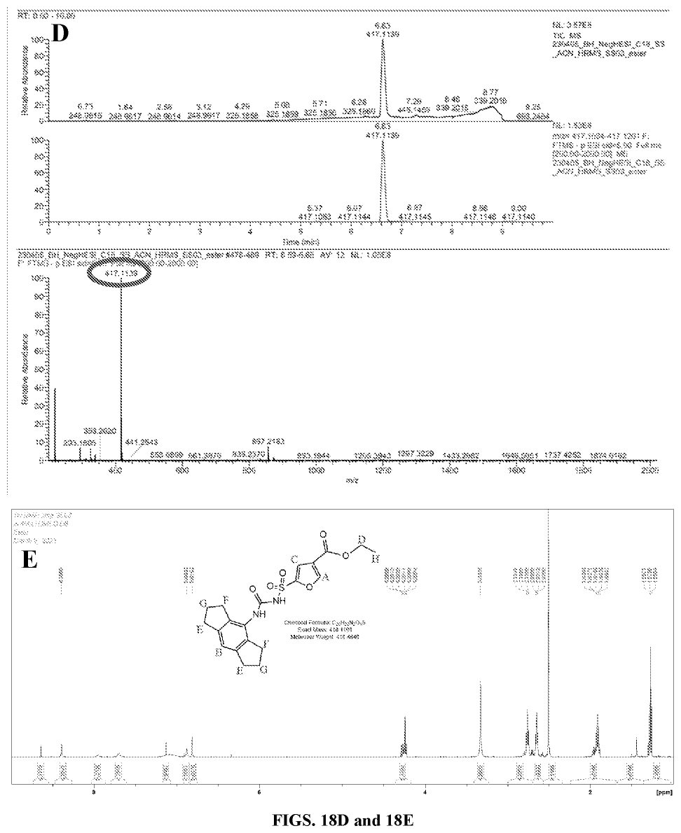

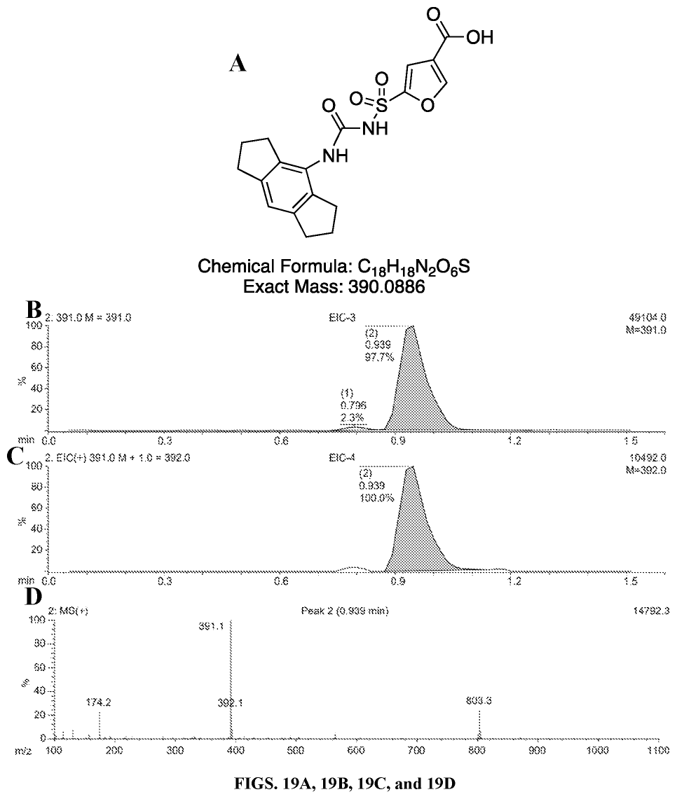

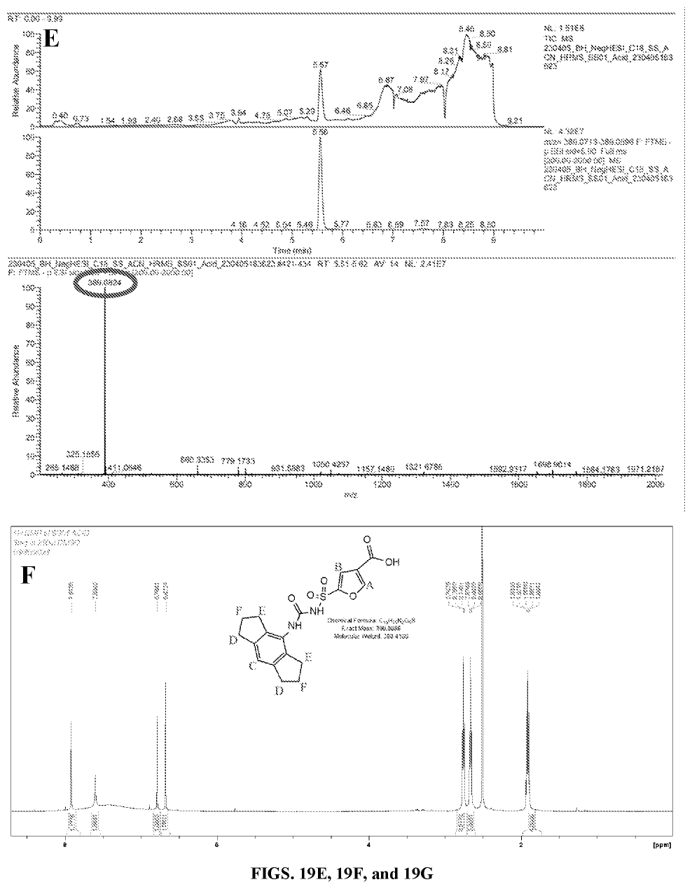

In some embodiments, the probes of any preceding aspect can be synthesized from a synthetic intermediate compound (See FIG. 16, 17, 18, or 19).

In some embodiments, the compounds or probes disclosed herein are used as an NLRP3 inhibitor. In some embodiments, the NLRP3 inhibitor is selected from:

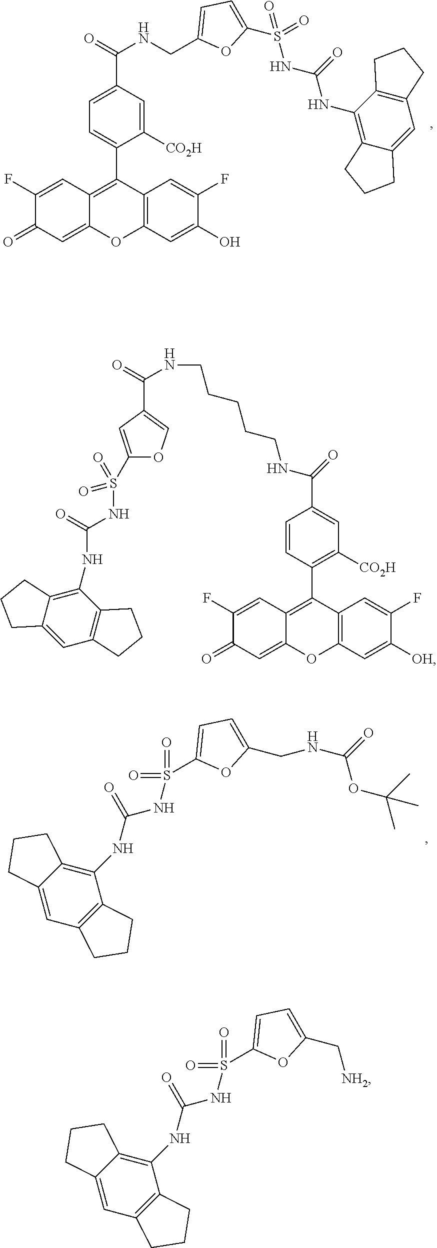 [SMILES: O=C(Nc1c2c(cc3c1CCC3)CCC2)NS(=O)(=O)c1ccc(CNC(=O)c2ccc(-c3c4cc(F)c(=O)cc-4oc4cc(O)c(F)cc34)c(C(=O)O)c2)o1 ; O=C(Nc1c2c(cc3c1CCC3)CCC2)NS(=O)(=O)c1cc(C(=O)NCCCCCNC(=O)c2ccc(-c3c4cc(F)c(=O)cc-4oc4cc(O)c(F)cc34)c(C(=O)O)c2)co1 ; CC(C)(C)OC(=O)NCc1ccc(S(=O)(=O)NC(=O)Nc2c3c(cc4c2CCC4)CCC3)o1 ; NCc1ccc(S(=O)(=O)NC(=O)Nc2c3c(cc4c2CCC4)CCC3)o1]

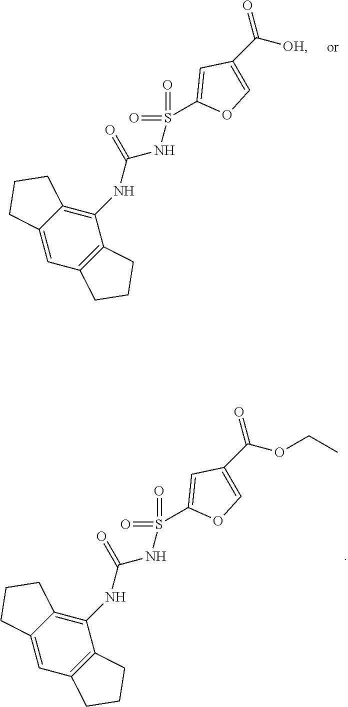 [SMILES: O=C(Nc1c2c(cc3c1CCC3)CCC2)NS(=O)(=O)c1cc(C(=O)O)co1 ; CCOC(=O)c1coc(S(=O)(=O)NC(=O)Nc2c3c(cc4c2CCC4)CCC3)c1]

### Methods

The present disclosure provides a method of detecting an ocular disease in a subject comprising administering to the subject a probe of any preceding aspect. As used herein “ocular disease” refers to a disease of the eye, including, but not limited to tumors, ocular degeneration, retinopathies, retinitis, retinal vasculopathies, diabetic retinopathies, diseases of the Bruch's membrane, or any combination thereof.

In some embodiments, the ocular disease is age-related macular degeneration (AMD), retinopathy of prematurity (ROP), diabetic retinopathy (DR), or branch retinal vein occlusion (BRVO).

As used herein, “age-related macular degeneration” (AMD) is a progressive vascular disease that mainly affects older adults and causes severe and irreversible vision loss. AMD can include a less severe AMD, “dry” AMD, or a more severe AMD, “wet” AMD. A complication that can contribute to wet AMD progression is choroidal neovascularization (CNV). Wet AMD symptoms can appear suddenly and worsen rapidly. Symptoms of wet AMD can include visual distortions, such as straight lines seeming bent, reduced central vision in one or both eyes, the need for brighter light when reading or doing close-up work, increased difficulty adapting to low light levels, such as when entering a dimly lit restaurant, increased blurriness of printed words, decreased intensity or brightness of colors, difficulty recognizing faces, a well-defined blurry spot or blind spot in your field of vision, or any combination thereof.

As used herein, “retinopathy of prematurity” (ROP) is an eye disorder caused by abnormal blood vessel growth in the light sensitive part of the eyes (retina) and can occur in the eyes of premature infants. In ROP, blood vessels can swell and overgrow in the light-sensitive layer of nerves in the retina at the back of the eye. When the condition is advanced, the abnormal retinal vessels can extend into the jellylike substance (vitreous) that fills the center of the eye. Bleeding from these vessels can scar the retina and stress its attachment to the back of the eye, causing partial or complete retinal detachment and potential blindness.

As used herein, “diabetic retinopathy” is a diabetes complication that affects eyes. It is caused by damage to the blood vessels of the light-sensitive tissue at the back of the eye (retina). Diabetic retinopathy can progress to a more severe type, known as proliferative diabetic retinopathy. In this type, damaged blood vessels close off, causing the growth of new, abnormal blood vessels in the retina. These new blood vessels are fragile and can leak into the clear, jellylike substance that fills the center of your eye (vitreous). Eventually, scar tissue from the growth of new blood vessels can cause the retina to detach from the back of your eye. If the new blood vessels interfere with the normal flow of fluid out of the eye, pressure can build in the eyeball. This buildup can damage the nerve that carries images from your eye to your brain (optic nerve), resulting in glaucoma.

As used herein, “branch retinal vein occlusion” is a blockage of one or more branches of the central retinal vein, which runs through the optic nerve. Branch Retinal Vein Occlusion symptoms include peripheral vision loss, blurred, or distorted central vision, floaters, or any combination thereof.

In some embodiments, the probe detects an activated NLRP3-mediated inflammasome. As used herein, the term “inflammasome” refers to cytosolic multiprotein oligomers of the innate immune system responsible for the activation of inflammatory responses. Activation and assembly of the inflammasome promotes proteolytic cleavage, and maturation and secretion of pro-inflammatory cytokines. As used herein, “an NLRP3-mediated inflammasome” is a multiprotein complex that initiates immune responses after being activated by a variety of stimuli, such as pathogens or cellular damage.

In some embodiments, the probe detects a pro-inflammatory macrophage. Proinflammatory macrophages, generally referred to as classically activated or M1-like, are responsible for killing pathogens and presenting their antigens to the adaptive immune system.

In some embodiments, the probe treats or prevents an ocular disease of any preceding aspect.

Also disclosed herein are methods of treating an ocular disease (as defined herein) in a subject including the step of detecting an ocular disease using the methods disclosed herein, and administering to the subject a suitable therapy for treating the disease. In certain implementations the subject is administered one or more therapeutic agents, receives radiation, undergoes a surgical procedure, or a combination thereof. In some embodiments, the probe may be administered to the subject subsequent to the therapy to determine whether the disease has been treated.

In one aspect, disclosed herein is a method for ocular imaging comprising administering to a subject the probe of any preceding aspect. Ocular imaging is used for diagnostic imaging of the posterior segment of the eye.

In some embodiments, the probe is administered to the subject in combination with a retinal imaging system. As used herein, “retinal imaging” refers to imaging that records the structural information of the retina. Types of retinal imaging include fundus photography, OCT, and fluorescein angiography. Fundus photography can be used for population-based, large-scale detection of DR, glaucoma, and AMD. OCT and fluorescein angiography can be used in daily management of patients in a retina clinic setting.

In certain embodiments, the probe is administered to the subject, and one or more ocular locations on the subject is irradiated at a wavelength from 450-600 nm, 450-500 nm, from 475-500 nm, from 475-525 nm, or from 500-525 nm.

The probe may be administered in such amounts, time, and route deemed necessary in order to achieve the desired result. The exact amount of the probe will vary from subject to subject, depending on the species, age, and general condition of the subject, the severity of the ocular disease, the particular probe, its mode of administration, its mode of activity, and the like. The probe is preferably formulated in dosage unit form for ease of administration and uniformity of dosage. It will be understood, however, that the total usage of the probe will be decided by the attending physician within the scope of sound medical judgment.

The probe may be administered by any route. In some embodiments, the probe is administered via a variety of routes, including oral, intravenous, intramuscular, intra-arterial, transdermal, interdermal, topical (as by powders, ointments, creams, and/or drops), mucosal, nasal, buccal. In general, the most appropriate route of administration will depend upon a variety of factors including the nature of the probe (e.g., its stability in the environment of the ophthalmic system), the condition of the subject (e.g., whether the subject is able to tolerate the chosen route of administration), etc.

The exact amount of the probe required to achieve an effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In some embodiments, the probe is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the probe is administered daily. In some embodiments, the probe is administered every day, every 2 days, every 3 days, every 4 days, every 5 days, every 6 days, every 7 days, or more. In some embodiments, the probe is administered every week, every 2 weeks, every 3 weeks, every 4 weeks, or more. In some embodiments, the probe is administered every month, every 2 months, every 3 months, every 4 months, every 5 months, every 6 months, every 7 months, every 8 months, every 9 months, every 10 months, every 11 months, every 12 months, or more. In some embodiments, the probe is administered every year, every 2 years, every 3 years, every 4 years, every 5 years, or more.

In one aspect, disclosed herein is a probe of any preceding aspect and a pharmaceutically acceptable carrier selected from an excipient, a diluent, a salt, a buffer, a stabilizer, a lipid, an emulsion, a nanoparticle, and a cream. One or more active agents can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

In some embodiments, the probe emits a fluorescent signal following exposure to a light stimulus. In some embodiments, the light stimulus is a laser source. As used herein, “laser source” can include, but is not limited to, argon lasers, diode lasers, micropulse lasers, or yttrium-aluminum-garnet (YAG) lasers.

As used herein, “inhibitors” or “antagonists” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is about 80%, optionally 50% or 25, 10%, 5%, or 1% or less.

In some embodiments, the probe comprises an NLRP3 inhibitor. In some embodiments, the NLRP3 inhibitor comprises an MCC-950 inhibitor. In some embodiments, the NLRP3 inhibitor comprises a CY-09 inhibitor.

As used herein, “NLRP3 inhibitors” are small molecule inhibitors of NLRP3 inflammasomes with the potential to decrease inflammation and inflammasome-mediated cell death. NLRP3 inhibitors include, but are not limited to, MCC950, ethyl 2-((2-chlorophenyl)(hydroxy)methyl)acrylate, CY-09 inhibitor, INF39, MNS, or OLT1177.

In some embodiments, the subject is a mammal.

The present disclosure also provides a method of inhibiting a NOD-, LRR-, and PYR-containing protein 3 (NLRP3) inflammasome comprising administering an effective amount of a probe, a compound, or an NLRP3 inhibitor of any preceding aspect.

The present disclosure also provides a method of inhibiting a NOD-, LRR-, and PYR-containing protein 3 (NLRP3) inflammasome in a cell or subject comprising administering to the cell or subject a probe, a compound, or an NLRP3 inhibitor of any preceding aspect.

The present disclosure also provides a method of treating or preventing an ocular disease in a subject comprising administering to the subject a probe, a compound, or an NLRP3 inhibitor of any preceding aspect. As used herein “ocular disease” refers to a disease of the eye, including, but not limited to tumors, ocular degeneration, retinopathies, retinitis, retinal vasculopathies, diabetic retinopathies, diseases of the Bruch's membrane, or any combination thereof.

In some embodiments, the ocular disease is age-related macular degeneration (AMD), retinopathy of prematurity (ROP), diabetic retinopathy (DR), or branch retinal vein occlusion (BRVO).

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1. Design and Synthesis of an Optical Imaging Probe, InflammaProbe-1, for Targeted Visualization of NLRP3 Inflammasomes in a Mouse Model of Age-Related Macular Degeneration

Age-related macular degeneration (AMD) is a progressive vascular disease that mainly affects adults older than 55 years and causes severe and irreversible visual impairments, accounting for about 6-9% of blindness worldwide. Early-stage AMD or dry AMD is the most common form, but almost all cases of dry AMD progress to a more severe condition called neovascular or wet AMD. While dry AMD is characterized by the formation of drusen in the sub-retinal space, wet AMD is distinguished by the pathological growth of abnormal choroidal blood vessels known as choroidal neovascularization (CNV). The progression of CNV may be critically regulated by vascular endothelial growth factor (VEGF), a protein that stimulates blood vessel growth. Predictably, anti-VEGF therapy is highly effective for management of wet AMD. However, many wet AMD patients do not respond favorably to anti-VEGF drugs, which presents a major challenge to clinicians who want to impede the advancement of CNV. Resistance to anti-VEGF treatments may reflect the existence of other important mediators of this disease. Indeed, the pathological progression of wet AMD is driven in part by leukocytes, such as activated monocytes, that migrate to the site of choroidal neovascular lesions where they become macrophages and secrete pro-inflammatory cytokines. The molecular mechanism of macrophage-mediated inflammation in CNV may involve the NOD-, LRR-, and PYR-containing protein 3 (NLRP3) inflammasome—a multiprotein complex that initiates immune responses after being activated by a variety of stimuli, such as pathogens or cellular damage. When NLRP3 is activated, it oligomerizes and binds to an adaptor protein, apoptosis-associated speck-like protein containing a CARD (ASC), and an effector protein, pro-caspase 1. Pro-caspase 1 is then cleaved to its enzymatically active form, caspase 1, which in turn activates pro-inflammatory cytokines, such as interleukin-1β (IL-1β), that are secreted by the cell. A study showed that IL-1β levels were increased 4-fold in the vitreous of patients with wet AMD in comparison to the control group.

Since NLRP3 is correlated with inflammation in CNV, visualizing NLRP3 inflammasomes and their associated macrophages is of great interest to clinicians and researchers who aim to monitor and study the progression of AMD. In turn, this may enable the development of effective therapies for patients who do not respond to anti-VEGF drugs. However, existing ophthalmic imaging systems do not permit targeted imaging of NLRP3 in activated macrophages. For instance, Optical Coherence Tomography (OCT) is a non-invasive and high-resolution imaging tool that is widely used to diagnose AMD, but it cannot effectively distinguish immune cells from retinal pigmented epithelial cells or other cells. A related tool, Adaptive Optics Scanning Laser Ophthalmoscopy (AO-SLO), was recently used to study the spatiotemporal dynamics of microglia in a mouse model of photoreceptor damage, but this method does not provide key information about the presence or absence of NLRP3 inflammasomes in microglia.

### Results

Design and synthesis of InflammaProbe-1

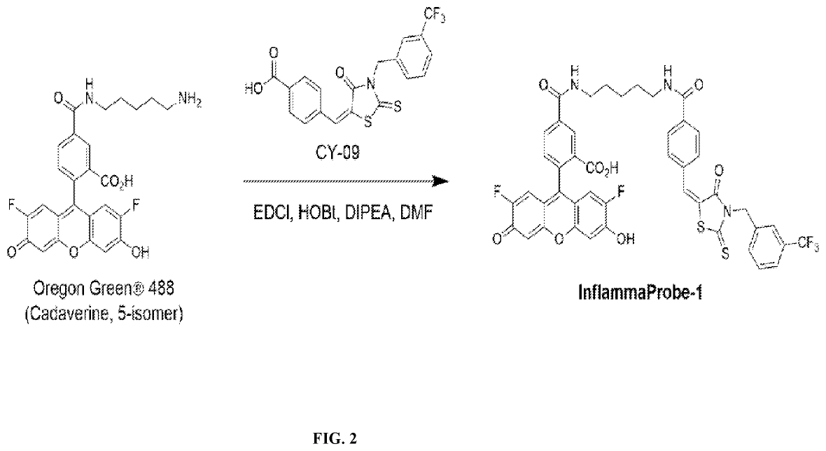

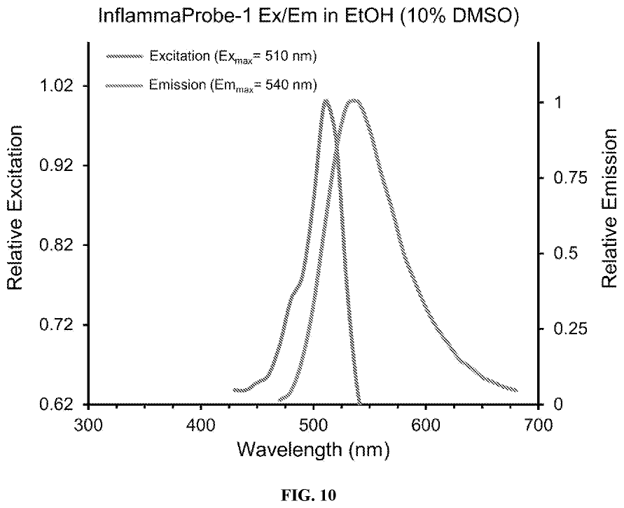

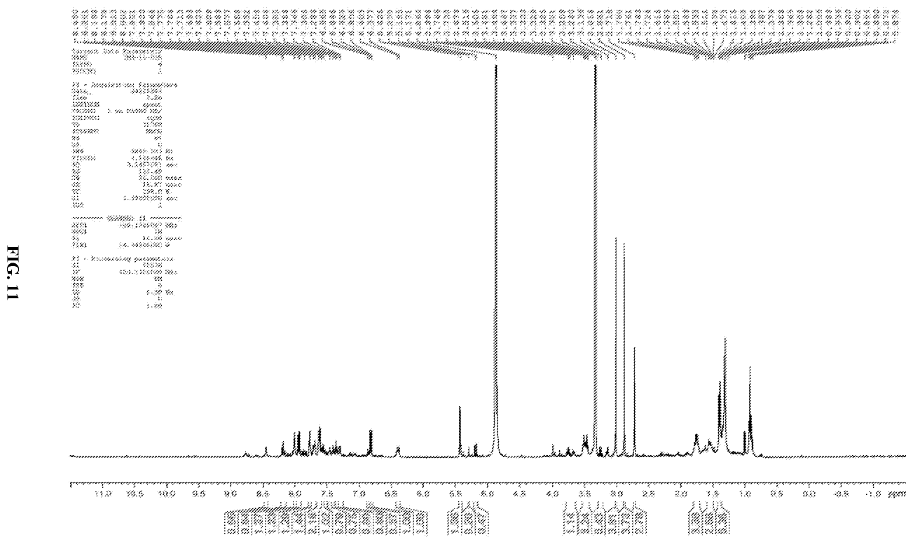

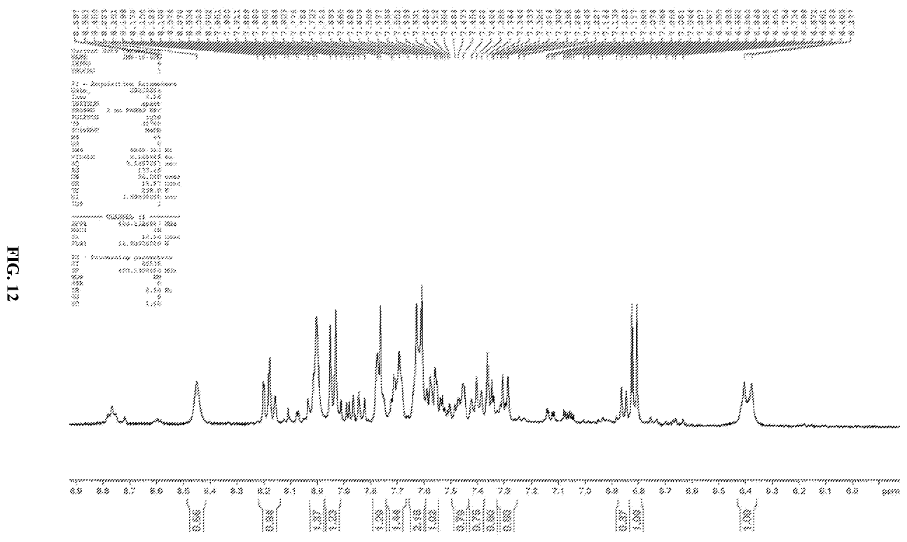

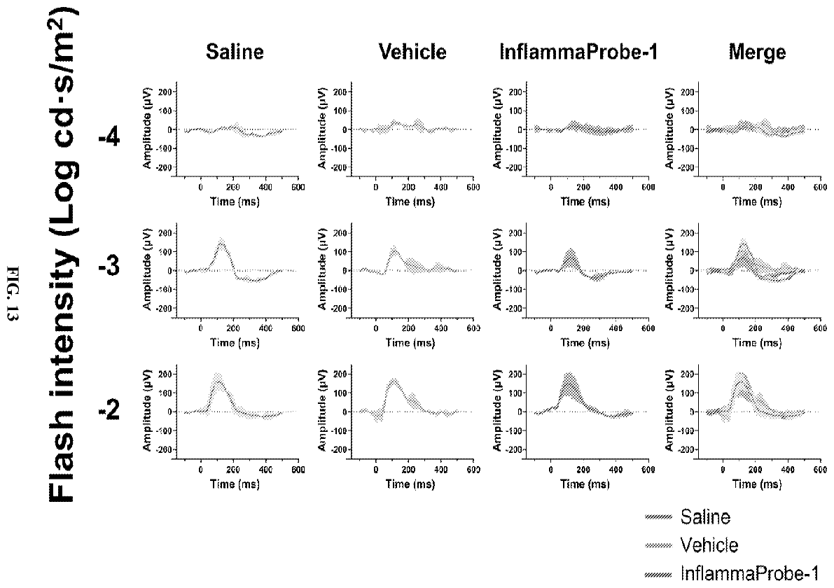

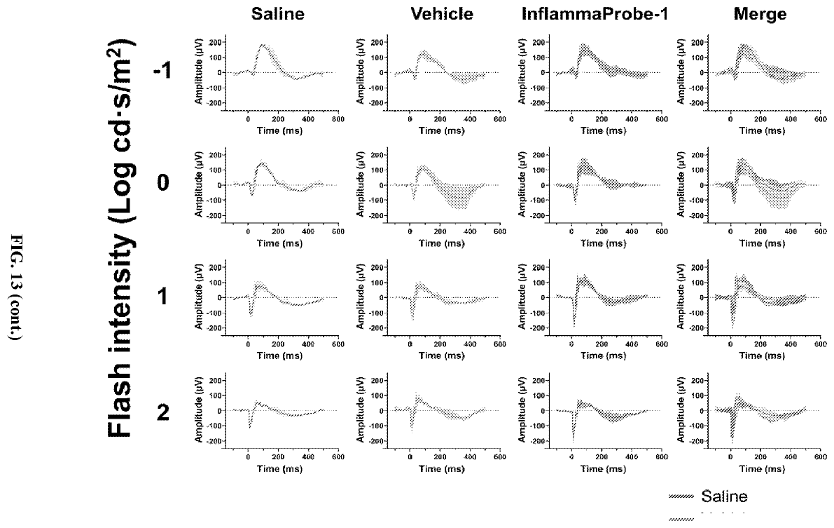

The NLRP3-targeted optical imaging probe, InflammaProbe-1, was designed by conjugating two components: 1) A fluorophore that would allow fluorescence-based visualization, and 2) a selective NLRP3 inhibitor that would enable targeting of NLRP3. With respect to the fluorophore, Oregon Green® 488, a bright and widely used dye derived from the FDA-approved fluorescein was chosen. Indeed, fluorescein angiography (FA) is a common imaging method used in most ophthalmic clinics. For the NLRP3 inhibitor, CY-09, a molecule that has been shown to inhibit NLRP3 selectively and directly by binding to its NACHT domain, was selected to block activation of the NLRP3 inflammasome. When tested in a murine model, CY-09 suppressed NLRP3-mediated cellular secretion of IL-1β and alleviated inflammatory disorders.¹⁶ Therefore, InflammaProbe-1 was synthesized by conjugating the selective inhibitor of NLRP3, CY-09, to the fluorophore, Oregon Green® 488 (FIG. 2). A spectroscopic analysis of InflammaProbe-1 was consistent with its predicted mass (FIG. 9) and a spectral scan showed its excitation/emission (ex/em) maxima at 510/540 nm (FIG. 10). A slight redshift of the ex/em maxima was observed relative to Oregon Green® 488, possibly due to conjugation with CY-09.

### Inflammaprobe-1 Targets the NLRP3 Inflammasome

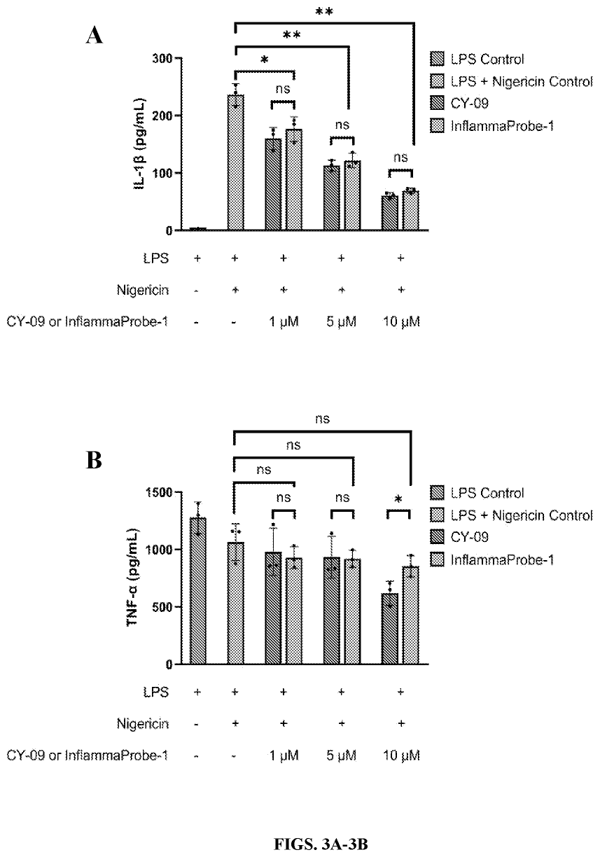

Following synthesis and characterization, InflammaProbe-1's ability to target NLRP3 was confirmed by comparing its inhibitory ability to that of its parent compound, CY-09, using enzyme-linked immunosorbent assay (ELISA). As shown in FIG. 3a, both CY-09 and InflammaProbe-1 dose-dependently inhibited NLRP3-mediated secretion of IL-1β in LPS-primed and nigericin-stimulated murine bone marrow-derived macrophages (BMDM). InflammaProbe-1 was just as effective as CY-09 at inhibiting NLRP at all three concentrations that were tested. At 1, 5 and 10 μM, both compounds caused a ˜1.3-fold decrease (p<0.05), ˜2-fold decrease (p<0.01), and ˜4-fold decrease (p<0.01), respectively, of IL-1β levels in comparison to the LPS and nigericin control. To test for an undesired off-target effect, the cell supernatants were also assayed for tumor necrosis factor-α (TNF-α). InflammaProbe-1 had no significant effect on LPS-induced secretion of TNF-α in comparison to the LPS and nigericin control (FIG. 3b). CY-09 showed similar results at 1 and 5 μM, but not at 10 μM; at this concentration, CY-09 significantly decreased the levels of TNF-α in comparison to InflammaProbe-1 (p<0.05). Overall, these results show that InflammaProbe-1 retains the inhibitory properties of CY-09 that enable it to target the NLRP3 inflammasome.

### In Vitro Imaging of NLRP3 in Activated BMDMs

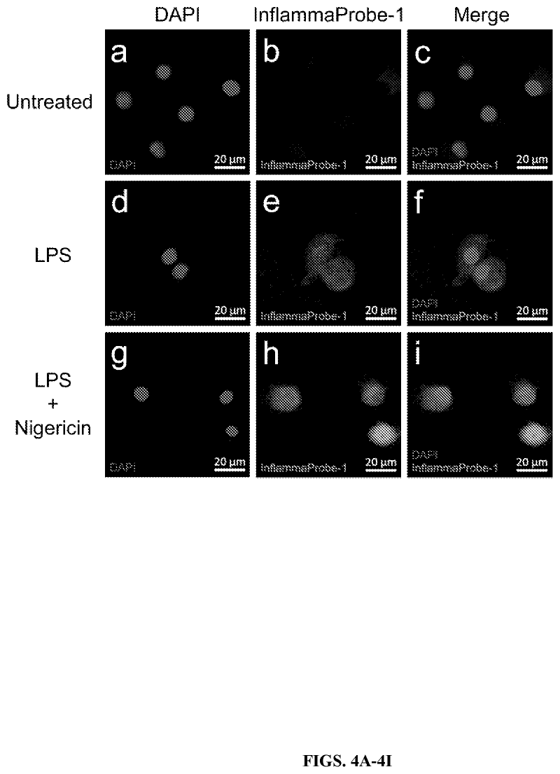

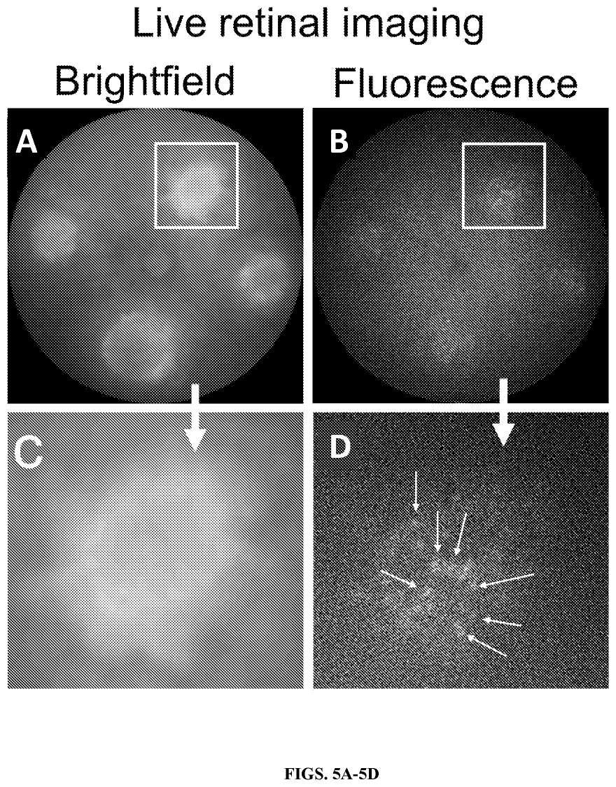

After confirming InflammaProbe-1's ability to target NLRP3, it was used to visualize NLRP3 in activated macrophages in vitro. Three different groups of BMDMs-untreated BMDMs, LPS-primed BMDMs, and LPS-primed and nigericin-stimulated BMDMs-were stained with InflammaProbe-1, fixed on microscope slides, and imaged using confocal fluorescence microscopy. Untreated BMDMs showed no remarkable InflammaProbe-1-dependent fluorescence (FIG. 4a-c). In contrast, LPS-primed BMDMs displayed appreciable fluorescence primarily in the cytosol (FIG. 4d-f). Thus, localization of NLRP3 was achieved in activated BMDMs using InflammaProbe-1. A similar pattern was observed in LPS-primed and nigericin-stimulated BMDMs, which showed even brighter fluorescence (FIG. 4g-i). These results show that InflammaProbe-1 could selectively stain macrophages that have been induced to express NLRP3.

### In Vivo Imaging of NLRP3 in LCNV

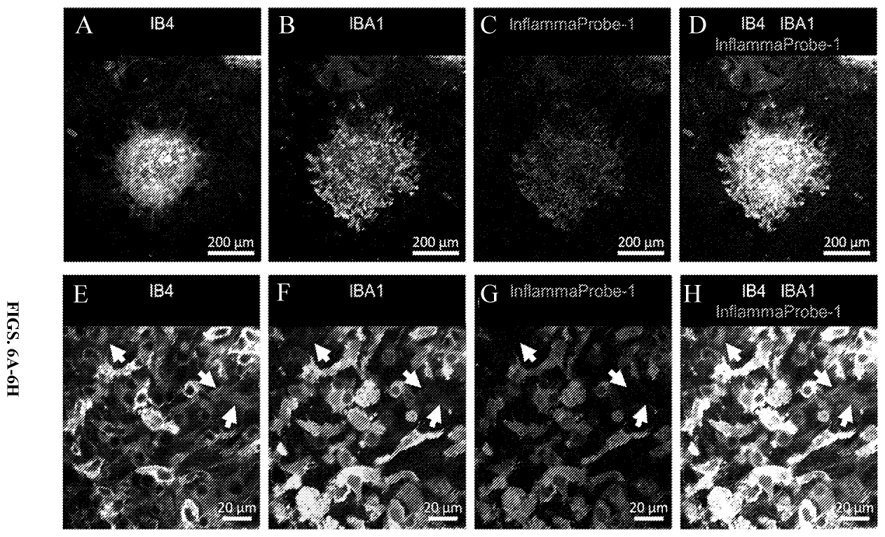

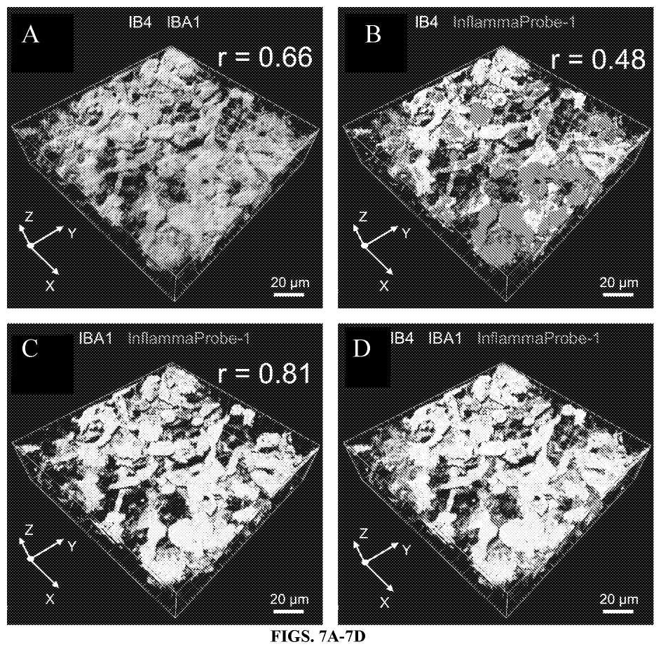

Next, InflammaProbe-1 was used to visualize NLRP3 inflammasomes in murine laser-induced choroidal neovascularization (LCNV), a well-established model of wet AMD. Four days post induction of LCNV, inflammaProbe-1 was injected intraperitoneally into mice. Six hours post injection, brightfield and fluorescence fundus images clearly showed cellular localization of InflammaProbe-1-dependent fluorescence to each of the four CNV lesions (FIG. 5). InflammaProbe-1 was not detected in any of the non-lesioned regions, which served as the healthy controls. Notably, the density of InflammaProbe-1-positive cells was higher at the center of the lesions than the periphery. This is the first evidence that NLRP3 inflammasomes and their associated activated cells can be visualized in a living ocular disease model.

### Ex Vivo Imaging of NLRP3 in LCNV

Following in vivo imaging of NLRP3 in murine LCNV, InflammaProbe-1's specificity for macrophages was investigated by comparing the colocalization of InflammaProbe-1, macrophages, and another prevalent cell type at the choroidal neovascular lesion—endothelial cells. To do so, the dissected choroids were co-stained with fluorescently tagged antibodies against ionized calcium binding adaptor molecule 1 (IBA1)—a selective marker for microglia/macrophages—and Isolectin B4 (IB4)—a marker that targets endothelial cells, but which has also been shown to stain macrophages. Confocal fluorescence imaging of the stained choroidal lesion at 10× magnification revealed substantial correlation between InflammaProbe-1 and macrophages (cells that were IBA1+; FIG. 6b-d). InflammaProbe-1 and its associated macrophages were dispersed throughout the lesion, including the center and periphery. In contrast, endothelial cells (cells that were IB4⁺ but IBA1⁻) were localized to the center of the lesion (FIGS. 6a and 6d). At 63× magnification, InflammaProbe-1 had stained macrophages but not endothelial cells, which are shown by white arrows in FIG. 6e-h.

These observations were verified by conducting a quantitative correlation analysis on a three-dimensionally constructed model of the stained choroidal lesion (FIG. 7). There was a high degree of correlation between InflammaProbe-1 and IBA1, which stains only macrophages (r=0.81; FIG. 7c). The correlation was reduced by almost half when comparing InflammaProbe-1 and IB4, which stains primarily endothelial cells (r=0.48; FIG. 7b); although an r value of 0.48 still represents a moderate degree of correlation, it is likely accounted for by the fact that IB4 may also stain macrophages, which were highly associated with InflammaProbe-1. This explanation is consistent with the high degree of correlation found between IBA1 and IB4 (r=0.66; FIG. 7a) although it was still lower than the correlation between IBA1 and InflammaProbe (r=0.81). Overall, these data show that InflammaProbe-1 targets not endothelial cells, but NLRP3-associated macrophages.

### Toxicity of Inflammaprobe-1

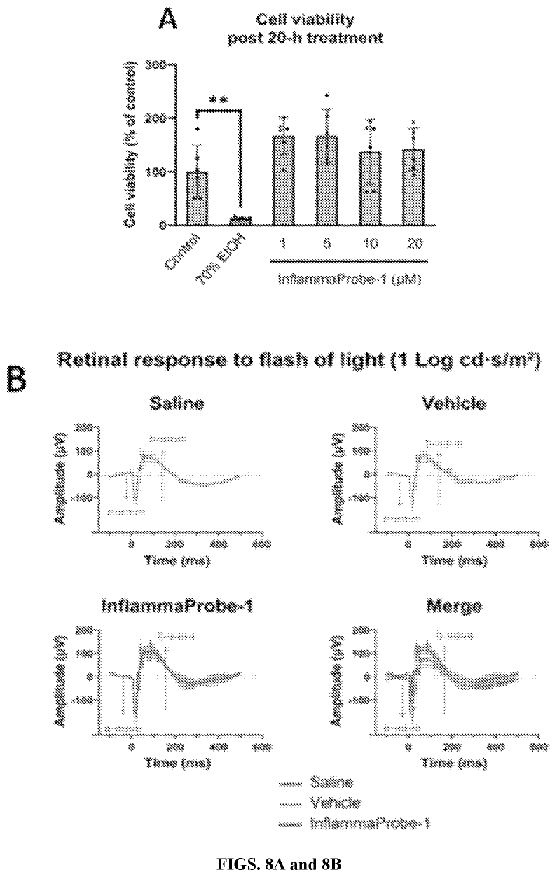

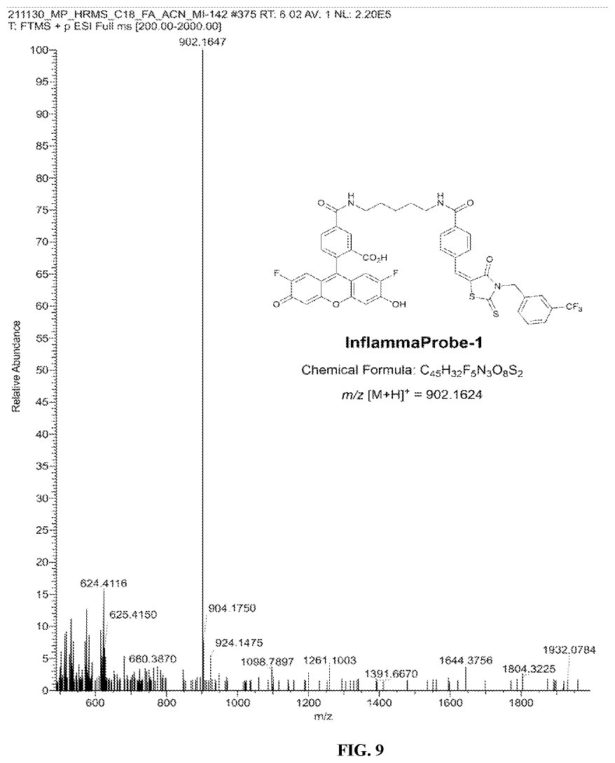

Finally, the toxicity of InflammaProbe-1 on primary cells and retinal tissues was assessed through in vitro and in vivo assays, respectively. To assess the probe's cytotoxicity in vitro, a Calcein Deep Red AM™ assay was performed on primary mouse retinal microvascular endothelial cells (MRMECs). As shown in FIG. 8a, a 20-h exposure of InflammaProbe-1 at up to 20 μM did not significantly reduce the viability of MRMECs in comparison to the untreated control group. In contrast, the positive control of 70% ethanol significantly decreased cell viability (p<0.01). Next, in vivo retinal toxicity was assessed in mice 7 days after an IP injection of InflammaProbe-1. Using dark-adapted, ganzfeld electroretinography (ERG), the mice's retinas were stimulated with flashes of light and electrical response evaluated. Specifically, analyses of the amplitude of the initial hyperpolarizing a-wave—which originates from photoreceptors—and the subsequent depolarizing b-wave—which is produced by cells that are post-synaptic to photoreceptors, including muller cells and on-bipolar cells, was conducted. As shown in FIG. 8b and FIG. 13, the retinas of mice injected with InflammaProbe-1 did not show any significant reduction in the a-wave and b-wave amplitudes relative to the retinas of mice injected with saline or a vehicle control. Based on these assays, InflammaProbe-1 does not appear to be toxic to primary cells or retinal tissues.

### Discussion

In this study, an NLRP3-targeted optical imaging probe enabled imaging of NLRP3 inflammasomes in activated macrophages and living ocular tissues. To do so, InflammaProbe-1 was first synthesized by conjugating the fluorophore, Oregon Green® 488, to the selective NLRP3 inhibitor, CY-09. Next, its ability to target NLRP3 was confirmed and used to visualize NLRP3 in LPS-primed and nigericin-stimulated murine macrophages. Then, using InflammaProbe-1, in vivo imaging of NLRP3 inflammasomes in LCNV, a murine model of wet AMD, was performed. This is the first evidence of in vivo molecular imaging of NLRP3 inflammasomes achieved in an ocular disease model. Subsequent ex vivo imaging of stained choroidal neovascular lesions confirmed substantial colocalization of InflammaProbe-1 and macrophages. Finally, InflammaProbe-1 did not appear to be toxic to primary cells or retinal tissues, as indicated by in vitro and in vivo cytotoxicity assays.

It should be noted that InflammaProbe-1 displayed slightly redshifted ex/em maxima relative to its parent fluorophore, Oregon Green® 488, which is why a 510 nm laser was used to achieve optimal in vitro and ex vivo visualization of NLRP3 inflammasomes. Nonetheless, a 488 nm laser was still able to capture high quality in vivo images of individual NLRP3-associated cells that had localized to CNV lesions.

It is reiterated that InflammaProbe-1 enabled in vivo imaging of NLRP3—an accomplishment that had not been previously reported in the literature. Based on these findings and on the well-known association between inflammation and wet AMD, this newly developed optical imaging technology can complement OCT and FA as a useful tool to study the onset, progression, and therapeutic response of wet AMD. It can also help answer questions about relevant molecular and cellular mechanisms involved in this vascular disease.

Finally, the utility of InflammaProbe-1 is not limited to AMD; its applications can be extended to other inflammatory diseases, such as proliferative diabetic retinopathy, that are mediated by the NLRP3 inflammasome.

### Methods

Reagents: All reagents were purchased and used as received unless otherwise indicated. Oregon Green® 488 Cadaverine, 5-isomer was obtained from Invitrogen (Waltham, MA). CY-09 was acquired from Tocris Bioscience (Bristol, UK) and resuspended in DMSO to make a 1 mM stock solution for experimental procedures. Rough strain lipopolysaccharides (LPS) from E. coli and nigericin sodium salt were purchased from Sigma-Aldrich (St. Louis, MO) and reconstituted to generate stock solutions of 1 mg/mL in PBS and 1 mM in ethanol, respectively. Rabbit anti-IBA1 antibody (Catalog No. ab 178847) was obtained from Abcam (Cambridge, UK) and Alexa Fluor® 594-conjugated anti-rabbit secondary antibody (Catalog No. A32754) was purchased from Invitrogen (Waltham, MA). DyLight® 649-conjugated GSL I Isolectin B4 (IB4) was acquired from Vector Laboratories (Burlingame, CA). HPLC grade solvents were obtained from Fisher Scientific (Waltham, MA). All other reagents, including deuterated solvents, were purchased from Sigma-Aldrich (Milwaukee, WI).

Synthesis and characterization of InflammaProbe-1: CY-09 (11.8 μmol), 1-hydroxybenzotriazole hydrate (HOBt; 11.8 μmol), N, N-diisopropylethylamine (DIPEA; 11.8 μmol), and 1-(3-dimethylaminopropyl)-3-ethylcarbodiimide hydrochloride (EDCI; 11.8 μmol) were added to a stirred solution of Oregon Green® 488 Cadaverine, 5-isomer (10.07 μmol) in dimethyl formamide (DMF; 2 mL) at 25° C. The resultant mixture was stirred for 2 days at 25° C. Removal of the solvent in vacuo afforded a residue that was purified by silica gel column chromatography using CHCl₃:MeOH:NH₄OH (35:7:1) to give an orange solid. InflammaProbe-1 was dissolved in DMSO to a 1 mM stock solution for experimental procedures, unless otherwise indicated. HRMS (ESI+): m/z [M+H]⁺ calcd for C₄₅H₃₂F₅N₃O₈S₂, 902.1624; Found, 902.1647 (Figure S1). See Supplementary Methods for additional details pertaining to chemical synthesis and characterization. The synthesis scheme was created with ChemDraw Professional (V20.1.1; PerkinElmer Informatics, Inc., Waltham, MA).

Animals: C57BL/6 mice, 4-6 weeks of age, were obtained from Charles River Laboratories (Wilmington, MA). At the time of the in vivo imaging and electroretinography studies, the mice were 12 and 14 weeks old, respectively. On average, male mice weighed 24 g and female mice weighed 21.5 g.

C57BL/6 mice were group-housed in ventilated cages according to their experimental group, and were maintained under a 12 h: 12 h light: dark cycle at 22±2° C. in an institutional animal care facility. They were provided clean water and a standard diet with 4.5% fat (PicoLab® Rodent Diet 5LOD; LabDiet St. Louis, MO) ad libitum. Mice were humanely sacrificed by CO₂ inhalation followed by cervical dislocation.

All animal procedures were approved by the Vanderbilt University Institutional Animal Care and Use Committee and were in accordance with the ARVO Statement for the Use of Animals in Ophthalmic and Vision Research and in compliance with ARRIVE guidelines.

Cell culture: Bone marrow-derived macrophages (BMDM), isolated from adult C57BL/6 mouse bone marrow, were purchased from ScienCell Research Laboratories (Carlsbad, CA) and cultured in ScienCell's phenol red-free Macrophage Medium (MaM) supplemented with 5% fetal bovine serum (FBS), Macrophage Growth Supplement (MaGS), and 1% Penicillin-Streptomycin (Pen, 100 U/mL; Strep, 100 μg/mL). Mouse Primary Retinal Microvascular Endothelial Cells (MRMEC), isolated from C57BL/6 mice, were purchased from Cell Biologics Inc. (Chicago, IL) and cultured in Cell Biologics' phenol red-free Endothelial Cell Medium supplemented with 0.1% VEGF, 0.1% Heparin, 0.1% EGF, 0.1% ECGS, 0.1% Hydrocortisone, 2 mM L-Glutamine, and 1% Antibiotic-Antimycotic. The medium was additionally supplemented with 10% FBS (R&D Systems, Minneapolis, MN). BMDMs and MRMECs were incubated at 37° C., 5% CO₂, and 95% relative humidity.

Inhibition assay of IL-1β and TNF-α: BMDMs were seeded in 12-well plates at a density of 5×10⁴ cells per well. After an overnight incubation, the cells were primed with 50 ng/mL LPS for 3 h, treated with 1 to 10 UM InflammaProbe-1 or CY-09 for 1 h, and stimulated with 10 μM nigericin for another hour to induce NLRP3 activation, as described in the literature. Cell culture supernatants were assayed for mouse IL-1β and TNF-α by performing enzyme-linked immunosorbent assays (ELISA; Invitrogen, Waltham, MA) in accordance with the manufacturer's instructions. The data were expressed as the mean±SD of 3 replicates per group.

In vitro imaging of NLRP3 in BMDMs: BMDMs were seeded in 4-chamber slides (Thermo Fisher Scientific, Waltham, MA) at a density of 1×10⁵ cells per chamber. After an overnight incubation, the cells were primed with 50 ng/ml LPS for 3 h, treated with 10 μM InflammaProbe-1 for 1 h, and stimulated with 10 μM nigericin for another hour to induce NLRP3 activation, as described in the literature. Then, the cells were washed with PBS twice, fixed with 4% neutral buffered formalin (NBF) for about 2 minutes, and washed with PBS twice. Immediately, the chambers were removed from the microscope slides in accordance with the manufacturer's instructions. Cells were mounted with Prolong™ Diamond Antifade Mountant with DAPI (Invitrogen, Waltham, MA) and imaged through confocal fluorescence microscopy. The images were representative of 4 replicates per group.

In vivo imaging of NLRP3 in LCNV: Induction of laser-induced choroidal neovascularization (LCNV) was performed in six adult C57BL/6 mice, three of each sex, following published protocols. Briefly, following anesthetization and pupillary dilation, four laser-induced choroidal lesions were created in each eye by rupturing the Bruch's membrane with an Argon laser photocoagulator (blue-green light) mounted on a slit lamp (Space Coast Laser, Inc., Palm Bay, FL). Lesions were created using the following laser parameters: 100 μm spot size, 0.1 s duration, and 0.1 Watts. Four days later, the LCNV mice were injected intraperitoneally with 10 mg/kg InflammaProbe-1 in 100 μL PBS with 10% DMSO. Brightfield and fluorescent fundus images were acquired 6 h post injection using the Micron IV retinal imaging system (Phoenix Research Laboratories, Pleasanton, CA). Annotations were added to both images and the contrast of the fluorescent fundus images were increased by 40% using PowerPoint (Microsoft, Redmond, WA). The images were representative of 12 eyes.

Ex vivo imaging of NLRP 3 in LCNV: After in vivo imaging, the LCNV mice that had been IP injected with InflammaProbe-1 at 10 mg/kg were sacrificed and enucleated. Then, the choroids were dissected and co-stained with DyLight® 649-conjugated IB4 and Alexa Fluor® 594-conjugated antibody against anti-IBA1. See Supplementary Methods for more details on the immunostaining procedure. The stained tissues were mounted on microscope slides with Prolong™ Diamond Antifade Mountant with DAPI (Invitrogen, Waltham, MA) and imaged through confocal fluorescence microscopy. The images were representative of 12 choroids.

3-D reconstruction of stained LCNV lesion and correlation analysis: A 33 μm thick Z-stack of a stained LCNV lesion was captured at 63× magnification using confocal fluorescence microscopy. Then, the Z-stack was used to construct a 3-D model and calculate Pearson's correlation coefficients (r) with Imaris software (V9.8.0; Oxford Instruments, Abingdon, UK).

Confocal microscopy and image processing: Confocal fluorescence microscopy was performed using an LSM 710 inverted microscope (Zeiss™, Jena, Germany). Image acquisition was conducted using ZEN Black Edition (V2.4, SP1; Zeiss™, Jena, Germany). Images were processed uniformly and identically across control and experimental groups using ZEN Blue edition (V2.6; Zeiss™, Jena, Germany) and PowerPoint (V2112; Microsoft, Redmond, WA). Refer to Tables 1-3 for more details on microscope configurations and image processing steps. Microscopy experiments and image processing were conducted in accordance with recommendations for rigor and reproducibility established in the literature.

Cell viability assay: MRMECs (Passage 4) were seeded on sterile black 96-well plates in complete medium at a density of 1.5×10⁴ cells per well. When the cells reached 80% confluence, they were treated with 1 to 20 μM InflammaProbe-1 in complete medium or 70% ethanol in water as the positive control for 20 h. The cells that were treated with ethanol were seeded on an identical but separate plate to prevent ethanol vapor from affecting the other experimental groups. After treatment, the cells were washed with HBSS containing Ca²⁺ and Mg²⁺. Then, to assay cell viability, the cells were exposed to an HBSS solution containing 5 μM Calcein Deep Red™ AM ester (AAT Bioquest, Sunnyvale, CA), a non-fluorescent compound that is cleaved into a brightly fluorescent product (exₘₐₓ/emₘₐₓ=643/663 nm) by esterases within live cells. This fluorescent compound is redshifted relative to InflammaProbe-1 (exₘₐₓ/emₘₐₓ=510/540 nm), which is necessary to avoid interference from any InflammaProbe-1-dependent fluorescence during fluorometric measurements. The cells were incubated at 37° C. for 1 h. Finally, fluorometric measurements were performed at ex/em=620/660 nm using the Cytation 5 microplate reader (BioTek Instruments, Inc., Winooski, VT). Fluorescence intensities were plotted as the percentage of cell viability relative to the control group. The data were expressed as the mean±SD of 6 replicates per group.

Electroretinography: Healthy, adult C57BL/6 mice were injected intraperitoneally with InflammaProbe-1 at 10 mg/kg in 100 μL PBS with 10% DMSO, 100 μL PBS with 10% DMSO as the vehicle control, or 100 μL 0.9% saline as an additional control. Six days post injection, the mice were dark-adapted inside a ventilated box overnight. After dark-adaptation, in vivo retinal toxicity was assayed through electroretinography (ERG) in accordance with published methods. The study was performed in a dark room under dim red light to avoid disruption of the dark adaptation. The mice were first anesthetized with a 70 μL IP injection containing a 1:1:2 mixture of Ketamine (85.7 mg/kg; Hospira, Inc., Lake Forest, IL), Xylazine (17.9 mg/kg; Akorn, Inc., Lake Forest, IL), and 0.9% saline. Then, the pupils were dilated with a drop each of 0.5% tropicamide, (Sandoz, Basel, Switzerland) and 2.5% phenylephrine (Paragon BioTeck, Inc., Portland, OR). The corneas were numbed with a drop of 0.5% proparacaine (Akorn, Inc., Lake Forest, IL). Mice were placed on a warm stage to maintain physiological body temperature. Next, a circular gold electrode was placed around each cornea, a reference electrode was inserted subcutaneously between the eyes, and a ground electrode was inserted subcutaneously at the base of the tail. Before starting the measurements, two drops of 0.9% saline were placed on each eye for hydration and electrical conductivity. The retinas were stimulated with flashes of white light (6500K) ranging from −4 to 2 Log cd s/m² using the ganzfeld ColorDome™ (Diagnosys LLC, Lowell, MA). Electrical responses were recorded using Espion software (V6, Diagnosys LLC, Lowell, MA) and plotted as voltage amplitude over time. Data were expressed as the mean±SD of 4 retinas per group.

Statistical Analysis: Data were expressed as mean±SD. Statistically significant differences between groups were determined by conducting unpaired t-tests with Welch's corrections. Statistical significance was defined as p≤0.05 (*), p≤0.01 (**), p≤0.001 (***), and p≤0.0001 (****) Statistical analyses and graphing were performed using Prism software (V9.3.0, GraphPad, San Diego, CA).

TABLE 1
Microscope configuration for in vitro imaging at 63x.
Image Dimensions

| | |
|---|---|
| Scaling (per pixel) | 0.19 μm × 0.19 μm |
| Image size (pixels) | 512 × 512 |
| Image size (scaled) | 95.62 μm × 95.62 μm |
| Bit depth | 8 bit |

Acquisition Information

| | |
|---|---|
| Software | ZEN Black Edition (V2.4, SP1) |
| Microscope | Zeiss ™ LSM 710, Inverted, AxioObserver |
| Objective | Plan-Apochromat 63x/1.40 Oil DIC M27 |
| Beam splitter | MBS 458/514 |
| Acquisition mode | Sequential multichannel acquisition |

| | DAPI | InflammaProbe-1 |
|---|---|---|
| Excitation wavelength/laser | 405 nm: 2.0% | 514 nm: 2.0% |
| Emission wavelength | 462 nm | 537 nm |
| Detector type | PMT | PMT |
| Detector gain | Variable | 822.9 |

Image Processing

| Software | Processing steps |
|---|---|
| ZEN Blue edition (V2.6) | Pseudocolor, scale bar, annotations, merge |
| PowerPoint (V2112) | Brightness: +20%, Contrast: −20% |

TABLE 2
Microscope configuration for ex vivo imaging at 10x.
Image Dimensions

| | |
|---|---|
| Scaling (per pixel) | 1.66 μm × 1.66 μm |
| Image size (pixels) | 512 × 512 |
| Image size (scaled) | 850.19 μm × 850.19 μm |
| Bit depth | 8 bit |

Acquisition Information

| | |
|---|---|
| Software | ZEN Black Edition (V2.4, SP1) |
| Microscope | Zeiss ™ LSM 710, Inverted, AxioObserver |
| Objective | Fluar 10x/0.50 M27 |
| Beam splitter | MBS 633/561/488 |
| Acquisition mode | Sequential multichannel acquisition |

| | IB4 | IBA1 | InflammaProbe-1 |
|---|---|---|---|
| Excitation | 633 nm: 2.0% | 561 nm: 2.0% | 514 nm: 2.0% |
| wavelength/laser | | | |
| Emission | 697 nm | 579 nm | 563 |
| wavelength | | | |
| Detector type | PMT | PMT | PMT |
| Detector gain | 741 | 583 | 822.9 |

Image Processing

| Software | Processing steps |
|---|---|
| ZEN Blue edition (V2.6) | Pseudocolor, scale bar, merge |
| PowerPoint (V2112) | annotations |

TABLE 3
Microscope configuration for ex vivo imaging at 63x.
Image Dimensions

| | |
|---|---|
| Scaling (per pixel) | 0.26 μm × 0.26 μm |
| Image size (pixels) | 512 × 512 |
| Image size (scaled) | 134.95 μm × 134.95 μm |
| Bit depth | 8 bit |

Acquisition Information

| | |
|---|---|
| Software | ZEN Black Edition (V2.4, SP1) |
| Microscope | Zeiss ™ LSM 710, Inverted, AxioObserver |
| Objective | Plan-Apochromat 63x/1.40 Oil DIC M27 |
| Beam splitter | MBS 633/561/488 |
| Acquisition mode | Sequential multichannel acquisition |

| | IB4 | IBA1 | InflammaProbe-1 |
|---|---|---|---|
| Excitation | 633 nm: 2.0% | 561 nm: 2.0% | 514 nm: 2.0% |
| wavelength/laser | | | |
| Emission | 697 nm | 579 nm | 563 |
| wavelength | | | |
| Detector type | PMT | PMT | PMT |
| Detector gain | 524.2 | 517.3 | 787.8 |

Image Processing

| Software | Processing steps |
|---|---|
| ZEN Blue edition (V2.6) | Pseudocolor, scale bar, merge |
| PowerPoint (V2112) | annotations, arrows |

### Example 2. Chemical Synthesis and Characterization

Moisture-sensitive reactions were performed in oven-dried glassware under a positive pressure of nitrogen or argon. Air and moisture-sensitive compounds were introduced via syringe or cannula through a rubber septum. High-resolution mass spectrometry was performed with an LTQ Orbitrap XL™ hybrid FT mass spectrometer (Thermo Scientific, Waltham, MA). The excitation and emission spectra (FIG. 10) were obtained using a Cytation 5 microplate reader (BioTek Instruments, Inc., Winooski, VT).

### Example 3. Immunostaining of Choroidal Tissue for Ex Vivo Imaging

After in vivo imaging, all LCNV mice were sacrificed. Their eyes were enucleated and fixed in 10% neutral buffered formalin (NBF) overnight at 4° C. The following morning, the eyes were washed with PBS and kept in PBS at 4° C. for two days. Then, the choroids were dissected, washed with PBS, and blocked/permeabilized in a solution containing wash buffer (TBS, 0.05% sodium azide, 0.33% Tween 20, and 0.0033% Triton-X), 1% bovine serum albumin (BSA), 10% donkey serum, and 0.2% fish gelatin for 2 h at room temperature. They were then exposed to a solution containing wash buffer, 0.2% BSA, and anti-IBA1 antibody (1:500 dilution) and left on a shaker overnight at 4° C. The next morning, the choroids were washed in wash buffer twice for two minutes each time, exposed to a solution containing wash buffer, 0.2% BSA, DyLight® 649-conjugated IB4 (1:100 dilution) and Alexa Fluor® 594-conjugated anti-Rabbit antibody (1:100 dilution), and placed on a shaker for 2 h at room temperature. Then, they were washed with wash buffer twice for two minutes each time and mounted on microscope slides with Prolong™ Diamond Antifade Mountant with DAPI (Invitrogen, Waltham, MA) in preparation for imaging.

Unless defined otherwise, all technical and scientific terms used herein have the same meanings as commonly understood by one of skill in the art to which the disclosed invention belongs. Publications cited herein and the materials for which they are cited are specifically incorporated by reference.

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A probe comprising a compound having a formula: 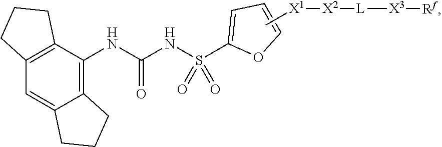
or a salt thereof,
wherein:
X¹ is selected from null, O, NH, C(═O), C(═O)NH, C(═O)O, NHC(═O)NH, NHC(═O)O or C₁₋₂alkyl;
X² is selected from null, O, NH, C(═O), C(═O)NH, C(═O)O, NHC(═O)NH, NHC(═O)O or C₁₋₂alkyl;
X³ is selected from null, O, NH, C(═O), C(═O)NH, C(═O)O, NHC(═O)NH, NHC(═O)O or C₁₋₂alkyl;
L is null or a linker; and
R^{f} is a fluorophore.

2. The probe of claim 1, comprising a compound having the formula:

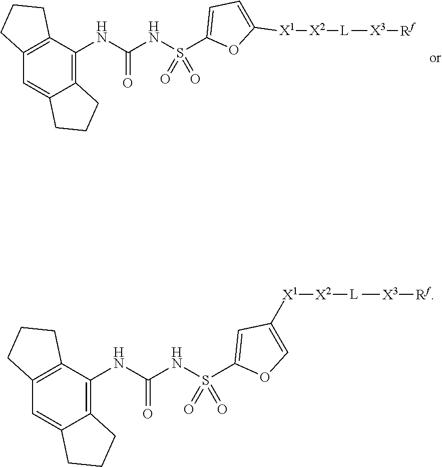

3. The probe of claim 1, wherein X¹ is null, CH₂, or C(CH₃)₂.

4. The probe of claim 1, wherein X¹ is null.

5. The probe of claim 1, wherein X¹ is CH₂.

6. The probe of claim 1, wherein X¹ is C(CH₃)₂.

7. The probe of claim 1, wherein X² is C(═O)NH or C(═O)O.

8. The probe of claim 1, wherein X² is C(═O)NH.

9. The probe of claim 1, wherein X² is C(═O)O.

10. The probe of claim 1, wherein L is null or C₁₋₈alkylene.

11. (canceled)

12. The probe of claim 1, wherein L is C₁₋₈alkylene.

13. The probe of claim 1, wherein L is C₂₋₆alkylene or C₄₋₆alkylene.

14. The probe of claim 1, wherein X³ is null, NH, O, C(═O)NH, C(═O)O, NHC(═O)NH, or NHC(═O)O.

15.-19. (canceled)

20. The probe of claim 1, wherein R^{f} is a xanthene fluorophore, BODIPY fluorophore, a cyanine fluorophore, a coumarin fluorophore, or an azo fluorophore.

21. The probe of claim 1, wherein R^{f} is a xanthene fluorophore selected from a rhodamine fluorophore, or a fluorescein fluorophore.

22. The probe of claim 1, wherein R^{f} has the formula: 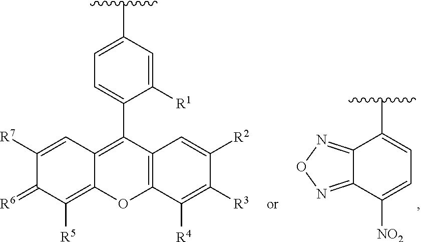
wherein
R¹ is H, CO₂H, or CO₂C₁₋₄alkyl;
R² is H or halo, e.g., F, Cl, Br, or I;
R⁴ is H or halo e.g., F, Cl, Br, or I
R⁵ is H or halo e.g., F, Cl, Br, or I
R⁷ is H or halo e.g., F, Cl, Br, or I
R³ is OH or N(Rⁿ)₂, wherein Rⁿ is in each case independently selected from H or C₁₋₄alkyl; and
R⁶ is O, N⁺(Rⁿ)₂, wherein Rⁿ is in each case independently selected from H or C₁₋₄alkyl.

23. The probe of claim 1, wherein R^{f} has the formula:

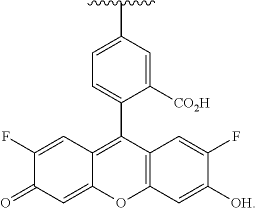 [SMILES: *c1ccc(-c2c3cc(F)c(=O)cc-3oc3cc(O)c(F)cc23)c(C(=O)O)c1]

24. The probe of claim 1, wherein R^{f} is fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.), ALEXA FLUOR™ 350, ALEXA FLUOR™ 405, ALEXA FLUOR™ 430, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, Pacific Orange, rhodamine 6G, rhodamine green, rhodamine red, tetramethyl rhodamine, Texas Red (available from Molecular Probes, Inc., Eugene, Oreg.), PE-Texas Red, APC-Cy7, PE-Alexa dyes (610, 647, and 680), or APC-Alexa dyes.

25. A method of detecting an ocular disease in a subject comprising administering to the subject the probe of claim 1.

26.-28. (canceled)

29. A method for ocular imaging comprising administering to a subject the probe of claim 1.

30.-35. (canceled)
